(19) United States
(12) Patent Application Publication
(10) Pub. No.: US 2026/0249088 A1
(43) Pub. Date: Aug. 27, 2026

(54) DELIVERY AND RETRIEVAL SYSTEM FOR A MEDICAL DEVICE

(71) Applicant: Medtronic, Inc., Minneapolis, MN (US)

(72) Inventors: Ronald A. Drake, St. Louis Park, MN (US); William J. Schindeldecker, Foreston, MN (US); Lester O. Stener, Hudson, WI (US)

(21) Appl. No.: 19/479,299

(22) PCT Filed: Mar. 28, 2024
(86) PCT No.: PCT/IB2024/053027

§ 371 Date: Oct. 27, 2025

Related U.S. Application Data
(60) Provisional application No. 63/498,956, filed on Apr. 28, 2023.

(51) Int. Cl. A61N 1/375; A61N 1/362; A61N 1/372

(52) U.S. Cl. CPC A61N 1/3758; A61N 1/37211; A61N 1/362; A61N 1/37512; A61N 1/37518; A61N 1/3756

(57) ABSTRACT

A medical system including a receptacle device configured to hold an implantable medical device (IMD) within a receptacle volume. The receptacle device is supported by a delivery catheter configured to position the receptacle device within an anatomical volume of a patient, such as a heart chamber. The receptacle device includes a receptacle electrode configured to conduct a signal from an environment surrounding the receptacle device to the receptacle volume such that, for example, the signal may communicate to an electrode positioned within the receptacle volume. The signal may be an intrinsic cardiac electrical signal produced by the patient's heart. The electrode may be configured to communicate the signal to processing circuitry for evaluation (e.g., by a clinician) of the positioning and/or proximity of the IMD relative to a target site, a suitability of the target site, a deployment of the IMD, and/or for other reasons.

This application claims the benefit of U.S. Provisional Patent Application Ser. No. 63/498,956, filed Apr. 28, 2023, the entire content of which is incorporated herein by reference.

### TECHNICAL FIELD

This disclosure is related to systems for delivery and/or retrieval of implantable medical devices.

### BACKGROUND

Various types of implantable medical devices have been implanted for treating or monitoring one or more conditions of a patient. Such implantable medical devices may be adapted to allow medical devices to monitor and/or treat conditions or functions relating to heart, muscle, nerve, brain, stomach, endocrine organs or other organs and their related functions. The implantable medical devices may be implanted at target locations selected to detect a physiological condition of the patient and/or deliver one or more therapies. For example, implantable medical devices may be delivered to locations within an atrium or ventricle of a heart to sense intrinsic cardiac signals and deliver pacing or antitachyarrhythmia shock therapy.

Some implantable medical devices are sized to be completely implanted within one of the chambers of the heart and/or another anatomical volume of the patient to detect a physiological condition and/or deliver one or more therapies. Such implantable medical devices may utilize delivery and/or retrieval systems to allow a clinician to navigate the implantable medical device (e.g., through vasculature of the patient) to the target location, and/or to retrieve the implantable medical device from the patient. In some examples, the implantable medical device may include one or more anchoring components intended to engage tissues at the target location (e.g., for implantation) and/or disengage from tissue at the target location (e.g., for retrieval).

### SUMMARY

The disclosure describes a medical system configured to deliver, position, retrieve, and/or otherwise re-orient an implantable medical device (“IMD”) within an anatomical volume (e.g., a chamber of a heart) within a patient. The medical system including a receptacle device configured to hold the IMD within a receptacle volume. The receptacle device includes a receptacle electrode configured to conduct a signal from an environment surrounding the receptacle device to the receptacle volume such that, for example, the signal may communicate to an electrode positioned within the receptacle volume.

For example, the receptacle device may be configured to conduct the signal to communicate the signal to a delivery system electrode supported by a delivery system engaged with the IMD, such that the delivery system electrode may communicate the signal to device processing circuitry supported by an external device extracorporeal to the patient. The receptacle device may be configured to conduct the signal to communicate the signal to one or device electrodes of the IMD, such that the device electrodes may communicate the signal to IMD processing circuitry supported by the IMD. In examples, the signal is an intrinsic cardiac electrical signal produced by the patient's heart. The electrode may be configured to communicate the signal to processing circuitry for evaluation (e.g., by a clinician) of the positioning and/or proximity of the IMD relative to a target site, a suitability of the target site, a deployment of the IMD, and/or for other reasons.

In an example, a medical system comprises: a receptacle device including a receptacle wall defining a receptacle volume configured to receive at least a portion of an implantable medical device, wherein the receptacle device is configured to be positioned within an anatomical volume defined by a body of a patient, and wherein the receptacle device includes an receptacle electrode configured to conduct a signal from an environment surrounding an exterior of the receptacle device to the receptacle volume; and a delivery catheter supporting the receptacle device, wherein the delivery catheter defines a delivery lumen and a delivery lumen opening which opens into the receptacle volume.

In an example, a method comprises: supporting, using a delivery catheter, a receptacle device, wherein the receptacle device includes a receptacle wall defining a receptacle volume configured to receive at least a portion of an implantable medical device, and wherein the delivery catheter defines a delivery lumen and a delivery lumen opening which opens into the receptacle volume; and conducting, using a receptacle electrode of the receptacle wall, a signal from an environment surrounding an exterior of the receptacle device to the receptacle volume.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical system and receptacle device within a heart.

FIG. 2 is a perspective illustration of an example receptacle device including a receptacle electrode.

FIG. 3 is a perspective illustration of an example receptacle device including an implantable medical device within a receptacle volume.

FIG. 4 is a cross-sectional schematic diagram of a receptacle device, with the cutting plane taken parallel to the page.

FIG. 5 is plan view of a portion of a delivery system engaging an implantable medical device.

FIG. 6 is plan view of a head portion of the delivery system of FIG. 5.

FIG. 7 is a cross-sectional schematic diagram of an example receptacle device with an implantable medical device in a first position, with the cutting plane taken parallel to the page.

FIG. 8 is a cross-sectional schematic diagram of an example receptacle device with an implantable medical device in a second position, with the cutting plane taken parallel to the page.

FIG. 9 is a cross-sectional schematic diagram of an example receptacle electrode, with the cutting plane taken parallel to the page.

FIG. 10 illustrates an example technique for conducting a signal using a receptacle electrode.

### DETAILED DESCRIPTION

This disclosure describes a medical system configured to deliver, position, and/or retrieve an implantable medical device (“IMD”) within an anatomical volume (e.g., a chamber of a heart) within a patient. In examples, the medical system is configured to deliver the IMD to a target site in the patient and convey signals (e.g., using processing circuitry) indicative of the positioning and/or proximity of the IMD relative to the target site, indicative of the suitability of the target site, indicative of the deployment of the IMD, and/or for other reasons.

The medical system includes a receptacle device (e.g., a device cup) configured to hold the IMD within a receptacle volume (e.g., a cup volume) during deployment of the IMD within the anatomical volume of the patient. The receptacle device is configured to conduct a signal from an environment surrounding the receptacle device (e.g., from fluids within the anatomical volume, and/or tissue within or surrounding the anatomical volume) to the receptacle volume such that, for example, the signal may communicate to a second electrode positioned within the receptacle volume (e.g., an electrode of the IMD within the receptacle volume and/or an another electrode within the receptacle volume). The signal may be an intrinsic cardiac electrical signal produced by the patient's heart. The second electrode may be configured to communicate the signal to processing circuitry for evaluation (e.g., by a clinician) of the positioning and/or proximity of the IMD relative to the target site, the suitability of the target site, the deployment of the IMD, and/or for other reasons.

In examples, the receptacle device includes a receptacle opening at a distal end configured to allow the IMD to pass therethrough during deployment of the IMD. The receptacle device may be configured such that the IMD may be translated (e.g., by a clinician) in a distal direction and/or proximal direction within the receptacle volume as, for example, the clinician position the IMD for deployment, evaluates the target site, and/or for other reasons. In examples, the receptacle device is supported at a proximal end by a delivery catheter defining a delivery lumen which opens into the receptacle volume. A clinician may translate the IMD within the receptacle volume using a delivery system extending through the delivery catheter to cause the IMD to at least partially extend through the receptacle opening during deployment of the IMD within the anatomical volume of the patient. In examples, the delivery system is configured such that the clinician may cause an attachment member of the IMD to engage tissues at the target site when the IMD at least partially extends through the receptacle volume.

In examples, the receptacle device is configured to conduct the signal from the environment surrounding the receptacle device to the receptacle volume as the IMD remains within the receptacle volume (e.g., during the deployment procedure). For example, the receptacle device may be configured to conduct the signal to the receptacle volume as the IMD is translated (e.g., by a clinician) within the receptacle volume during deployment of the IMD. Conducting the signal as the IMD remains positioned within the receptacle volume may reduce an extent to which the IMD needs to egress from the receptacle volume in order to receive sufficient signal for evaluation during the deployment. Reducing the degree of egress of the IMD which is necessary in order to evaluate a deployment may avoid a need to recapture the IMD with the receptacle device should the (e.g., degree of electrical contact established in) deployment be lacking, which may present some difficulty in relatively small anatomical chambers such as a heart chamber of the patient.

In examples, the receptacle device is configured to conduct the signal from the environment surrounding the receptacle device to the receptacle volume using a receptacle electrode. The receptacle electrode may be configured to define a conductive path through a receptacle wall defining the receptacle volume, such that a signal from an environment surrounding the exterior of the receptacle device may travel through the conductive path to the receptacle volume. In examples, the receptacle electrode is configured to substantially extend through the receptacle wall. For example, the receptacle wall may define an inner surface defining the receptacle volume and an outer surface configured to be in fluid communication with the environment surrounding the exterior of the receptacle device. In examples, the receptacle electrode extends from the outer surface to the inner surface. In examples, the receptacle electrode is substantially supported by and/or embedded within the receptacle wall.

In some examples, the receptacle electrode defines an inner electrode face facing toward the receptacle volume and an outer electrode face facing away from the receptacle volume (e.g., facing the environment surrounding the exterior of the receptacle device). The receptacle electrode may be configured to receive the signal (e.g., an intrinsic cardiac electrical signal) via the outer electrode face and communicate the signal to the receptacle volume using the inner electrode face. The receptacle volume may be configured to communicate the signal to a second electrode (e.g., an IMD electrode and/or a delivery system device electrode) using, for example, fluid within the receptacle volume.

The receptacle electrode may be configured such that the inner electrode face communicates the signal to the second electrode (e.g., the IMD electrode) as the IMD is translated (e.g., by a clinician) distally and/or proximally within the receptacle volume. In examples, the receptacle electrode is configured such that the inner electrode face extends longitudinally (e.g., proximally and distally) within the receptacle volume, such that the receptacle electrode may maintain and/or establish connectivity with the IMD electrode as the IMD is translated (e.g., by the clinician) to different positions within the receptacle volume. For example, the medical system may be configured to substantially maintain the IMD in a delivery position within the receptacle volume as the medical system transports the receptacle device and the IMD (e.g., through vasculature) to a target site in a patient. Once in the vicinity of the target site, the medical system (e.g., under the influence of clinician) may be configured to translate the IMD distally from the delivery position and toward the receptacle opening at the distal end of the receptacle device to deploy the IMD at the target site. The inner electrode face may be configured to maintain and/or establish connectivity with the IMD electrode when the IMD is in the delivery position and as the IMD is moved to other positions distal to the delivery position within the receptacle volume.

In examples, the receptacle device is configured to establish and/or maintain physical contact between the inner electrode face and the IMD electrode as the IMD is translated to and/or assumes different positions within the receptacle volume. In some examples, the receptacle device is configured to maintain a displacement between the inner electrode face and the IMD electrode. The displacement may reduce and/or eliminate signal noise and/or other signal perturbations that might result as the IMD translates and intermittently makes physical contact with the inner electrode face. In examples, the receptacle device includes one or more ridges extending toward the receptacle volume to maintain the displacement between the inner electrode face and the IMD electrode. The one or more ridges may be configured to allow some portion of a fluid within the receptacle volume to flow into the displacement between the inner electrode face and the IMD electrode, such that the portion of the fluid may communicate the signal from the inner electrode face to the IMD electrode.

In examples, the medical system includes a delivery system configured to engage the IMD. The delivery system may be configured to slidably translate and/or rotate with the delivery lumen of the delivery catheter, such that relative movement between the delivery system and the delivery catheter may cause the IMD to position within the receptacle volume and/or exit the receptacle volume (via the receptacle opening). The delivery catheter may be configured to transition through the vasculature of a patient, such that the delivery system and/or the IMD may be retrieved from and/or delivered to an anatomical volume of the patient (e.g., a heart chamber).

Hence, the medical system includes a receptacle device configured to hold an IMD during deployment of the IMD within an anatomical volume (e.g., a heart chamber). The receptacle device is configured to conduct a signal (e.g., an intrinsic cardiac electrical signal) from an environment surrounding the receptacle device to the receptacle volume such that, for example, the signal may communicate to a second electrode positioned within the receptacle volume. For example, the receptacle device may be configured to receive the signal using a receptacle electrode and communicate the signal from the receptacle electrode to an IMD electrode and/or a delivery system device electrode. IMD electrode and/or delivery system device electrode may be configured to communicate the signal to processing circuitry for evaluation (e.g., by a clinician) of the positioning and/or proximity of the IMD relative to the target site, the suitability of the target site, the deployment of the IMD, and/or for other reasons.

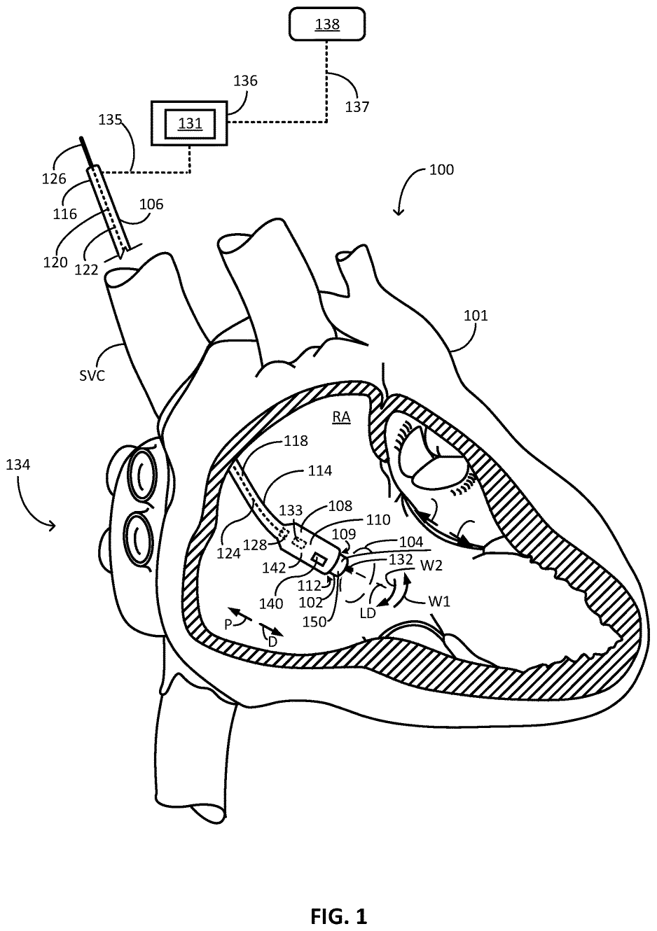

FIG. 1 is a conceptual diagram illustrating an example medical system 100 within a right atrium (“RA”) of a heart 101. Medical system 100 is configured to deliver and/or retrieve an implantable medical device 102 (“IMD 102”) to and/or from the vicinity of a target site 104 of heart 101. Medical system 100 includes a delivery catheter 106 supporting a receptacle device 108. Receptacle device 108 is configured to hold IMD 102 during delivery, deployment, and/or retrieval of IMD 102. Receptacle device 108 includes a receptacle wall 110 defining a receptacle volume 112 configured to hold and/or support IMD 102 during the delivery, deployment, and/or retrieval of IMD 102. In examples, receptacle device 108 defines an opening 109 (“receptacle opening 109”) which opens into receptacle volume 112. Receptacle opening 109 may be configured to allow at least IMD 102 to pass therethrough. In examples, medical system 100 is configured to deploy IMD 102 from receptacle device 108 (e.g., from a position within receptacle volume 112) and through receptacle opening 109 to cause IMD 102 to engage tissues within target site 104. In examples, medical system 100 is configured to cause IMD 102 to disengage from tissues within target site 104 to, for example, retrieve IMD 102 from and/or reposition IMD 102 within heart 101.

Delivery catheter 106 is configured to deliver receptacle device 108 and/or IMD 102 to an anatomical volume of the patient (e.g., the RA). Optionally, delivery catheter 106 may be advanced to the anatomical volume of the patient through a surrounding tubular member (not shown), such as a sheath or guide catheter, which may be placed with its distal end in the anatomical volume before delivery catheter 106 is advanced through the surrounding tubular member. In examples, delivery catheter 106 is configured to retrieve receptacle device 108 and/or IMD 102 from the anatomical volume of the patient. Delivery catheter 106 may include a distal portion 114 (“delivery catheter distal portion 114”) configured to be intracorporeal to the patient and a proximal portion 116 (“delivery catheter proximal portion 116”) which may be extracorporeal to the patient when delivery catheter distal portion 114 is intracorporeal. In examples, delivery catheter distal portion 114 supports (e.g., is attached to and/or is a substantially unitary component with) receptacle device 108. In examples, delivery catheter 106 is configured to deliver and/or retrieve receptacle device 108 and/or IMD 102 using vasculature of a patient, such as an IVC or other vasculature leading to the anatomical volume. In examples, delivery catheter 106 defines a lumen 118 (“delivery lumen 118”) which opens to receptacle volume 112.

In examples, medical system 100 includes a delivery system 120 configured to engage IMD 102 when, for example, IMD 102 is positioned within receptacle volume 112. Delivery system 120 includes a delivery system body 122 that may include a distal portion 124 (“delivery system body distal portion 124”) configured to be intracorporeal to the patient and a proximal portion 126 (“delivery system body proximal portion 126”) which may be extracorporeal to the patient when delivery system body distal portion 124 is intracorporeal. In examples, delivery system 120 includes a head portion 128 configured to engage IMD 102. At least delivery system body 122 may be configured to slidably translate and/or rotate within delivery lumen 118 (e.g., translate and/or rotate relative to delivery catheter 106). Delivery system 120 may be configured such that the translation and/or rotation of delivery system body 122 causes delivery system body 122 and/or head portion 128 to impart translational and/or rotational forces on IMD 102, such that IMD 102 translates and/or rotates relative to receptacle wall 110. For example, delivery system 120 may be configured to translate within delivery lumen 118 to impart (e.g., via head portion 128) a translational force on IMD 102 causing IMD 102 to translate (e.g., within receptacle volume 112) in the proximal direction P or the distal direction D relative to receptacle wall 110. Delivery system 120 may be configured to rotate within delivery lumen 118 to impart (e.g., via head portion 128) a rotational force on IMD 102 causing IMD 102 to rotate (e.g., within receptacle volume 112) about a device axis LD defined by IMD 102. In some examples, head portion 128 and delivery system body 122 may be substantially separate components. In some examples, head portion 128 may be substantially contiguous with delivery system body 122, such that head portion 128 and delivery system body 122 define a unified component. In FIG. 1, portions of delivery system body 122 and/or head portion 128 positioned within delivery lumen 118 and/or receptacle volume 112 are depicted with dashed lines.

Medical system 100 may be configured to position IMD 102 in proximity to target site 104 such that IMD 102 may be anchored to tissues within target site 104. In examples, IMD 102 includes an attachment member 132 configured to engage tissues within target site 104. For example, delivery catheter 106 may be configured (e.g., under the influence of a clinician) to traverse vasculature of the patient to position receptacle device 108 and IMD 102 in proximity to target site 104. Medical system 100 (e.g., delivery system body 122 and/or head portion 128) is configured to impart a torque to IMD 102 to cause attachment member 132 to engage tissues (e.g., tissue within target site 104) when attachment member 132 is within or in proximity to target site 104. In examples, medical system 100 (e.g., delivery system body 122 and/or head portion 128) is configured to impart a force (e.g., in the distal direction D) to IMD 102 to cause attachment member 132 to engage tissues (e.g., tissue within target site 104) when attachment member 132 is within or in proximity to target site 104. Medical system 100 may be configured such that delivery system body 122 and/or head portion 128 may be disengaged from IMD 102 as IMD 102 remains anchored to tissues within or in proximity to target site 104. Delivery catheter 106, receptacle device 108, and delivery system 120 may subsequently be withdrawn from the patient (e.g., via vasculature of the patient).

In examples, medical system 100 is configured to receive (e.g., sense) an indication of an intrinsic cardiac electrical signal produced by heart 101 when receptacle device 108 is positioned (e.g., by a clinician) within heart 101. Medical system 100 may be configured to process and/or condition a sensed signal (e.g., an intrinsic cardiac electrical signal) to provide, for example, an indication of a location of receptacle device 108 and/or IMD 102 within heart 101, to conduct pace mapping for deployment of IMD 102, to provide indications indicative of the deployment and/or attachment of IMD 102 within heart 101, or for other reasons. In examples, medical system 100 includes processing circuitry 134 configured to process and/or condition the sensed signal. In examples, processing circuitry 134 includes processing circuitry such as device processing circuitry 131, configured to be mechanically supported by an external device 136 and/or another device of medical system 100. External device 136 and/or device processing circuitry 131 may be configured to be extracorporeal to the patient and/or otherwise displaced from receptacle device 108 and/or IMD 102 when, for example, receptacle device 108 and/or IMD 102 are intracorporeal to the patient. In examples, processing circuitry 134 includes processing circuitry such as IMD processing circuitry 133, configured to be mechanically supported by a housing of IMD 102 and/or another device of medical system 100. IMD processing circuitry 133 may be configured to be intracorporeal to the patient when, for example, receptacle device 108 and/or IMD 102 are intracorporeal to the patient.

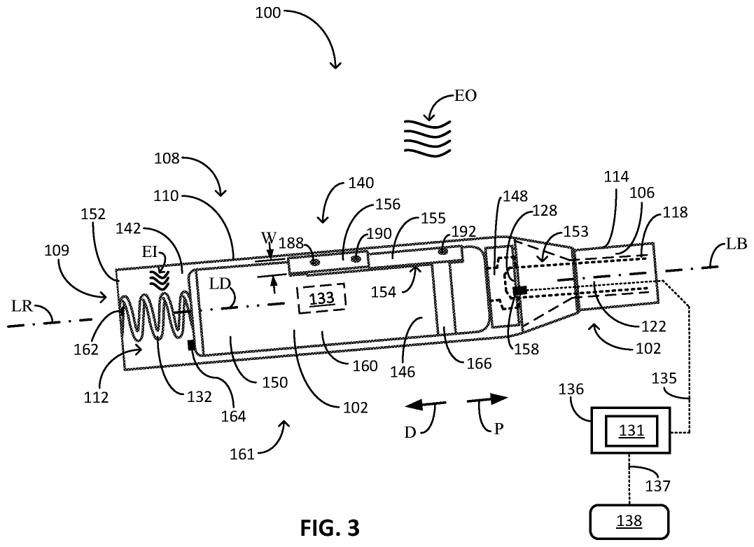

Processing circuitry 134 may be configured to process and/or condition the signal sensed by medical system 100 using at least a first electrode (e.g., one of a cathode or an anode) and a second electrode (e.g., the other of the cathode and the anode) in electrical communication with processing circuitry 134. For example, in some examples, the first electrode or second electrode is a delivery system electrode (e.g., delivery system electrode 158 (FIG. 3)) mechanically supported by delivery system body 122 and/or head portion 128. In some examples, the first electrode or second electrode is a device electrode (e.g., IMD return electrode 166 (FIG. 3)) mechanically supported by a housing of IMD 102, or one or more electrodes mechanically supported by another portion of medical system 100, such as receptacle device 108, delivery catheter 106, or another portion. In some examples, the first electrode or second electrode is an external electrode 138 configured to be extracorporeal to the patient and/or otherwise displaced from receptacle device 108 and/or IMD 102 (e.g., a cutaneous electrode affixed to the skin of the patient). Processing circuitry 134 may be configured to use the first electrode, the second electrode, and/or one or other electrodes of medical system 100 to receive the signal sensed by medical system 100. In examples, medical system 100 is configured to communicate the signal from at least the first electrode and/or the second electrode using one or more communication links, such as communication link 135 between a delivery system electrode of delivery system 120 and processing circuitry 134 and/or communication link 137 between external electrode 138 and processing circuitry 134. In some examples, for example when IMD 102 mechanically supports processing circuitry 134, IMD 102 may include one or more of the communication links.

In some examples, when a first electrode is positioned within receptacle volume 112, receptacle wall 110 and/or a body of IMD 102 may reduce reception of a signal (e.g., an intrinsic cardiac electrical signal) by a first electrode and/or second electrode. For example, when a first electrode is positioned within receptacle volume 112, receptacle wall 110 and/or the body of IMD 102 may act to at least partially shield the first electrode from adequately receiving the signal, act to attenuate the signal, and/or act to otherwise interfere with the signal as the signal travels over a signal path to the first electrode. In some cases, receptacle wall 110 and/or the body of IMD 102 may at least partially block a signal path from a fluid environment surrounding receptacle device 108 to the first electrode positioned within receptacle volume 112, such that receptacle wall 110 and/or the body of IMD 102 act to at least partially reduce a signal strength of the signal arriving at the first electrode. As an example, when a first electrode within receptacle volume 112 (e.g., a delivery system electrode of delivery system 120 and/or a device electrode of IMD 102) is generally reliant on a signal path proceeding through receptacle opening 109 for receipt of a signal, portions of IMD 102 positioned substantially between receptacle opening 109 and the first electrode may act to attenuate, at least partially shield, and/or otherwise interfere with the signal prior to reception by the first electrode. The attenuation, shielding, and/or interference of the signal may reduce an ability of processing circuitry 134 to process and/or condition the sensed signal.

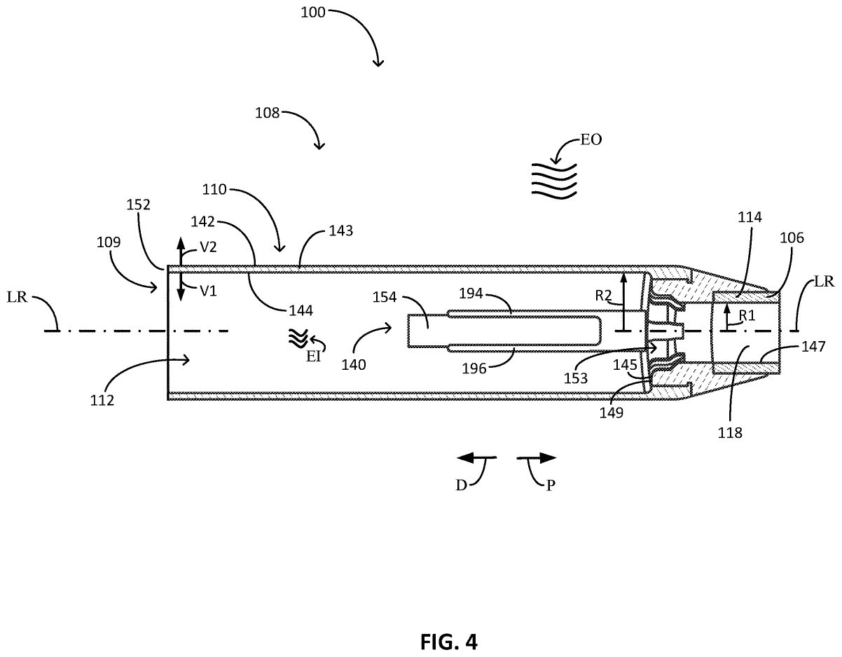

Receptacle device 108 is configured to provide a conductive path through receptacle wall 110 to reduce signal reductions (e.g., signal strength reductions by attenuation, shielding, and/or interference) which might otherwise occur over a signal path from an environment surrounding an exterior of receptacle device 108 to a first electrode within receptacle volume 112. Receptacle device 108 includes a receptacle electrode 140 configured to conduct a signal (e.g., an intrinsic cardiac electrical signal) from the environment surrounding receptacle device 108 (e.g., a fluid environment of a heart chamber of heart 101) to an electrode of medical system 100 positioned within receptacle volume 112. For example, receptacle electrode 140 may be configured to conduct the signal from the fluid environment of the heart chamber to a delivery system electrode of delivery system 120 and/or a device electrode of IMD 102. In some examples, receptacle electrode 140 is substantially embedded within receptacle wall 110. In some examples, receptacle electrode 140 extends from an outer surface 142 of receptacle wall 110 (“wall outer surface 142”) to an inner surface of wall 110 (“wall inner surface 144” (FIG. 4)). Wall outer surface 142 may be configured to fluidly communicate with the environment surrounding receptacle device 108, such as a fluid environment within a chamber of heart 101. The wall inner surface may define at least portion of a boundary of receptacle volume 112. In examples, receptacle wall 110 comprises a first material having a first conductivity and receptacle electrode 140 comprises a second material having a second conductivity greater than the first conductivity, such that electrode 140 defines a conductive path through receptacle wall 110.

In some examples, the conductive path provided by receptacle electrode 140 may improve an ability to determine (e.g., using processing circuitry 134) a location of receptacle device 108 and IMD 102 during a delivery of IMD 102 to a chamber of heart 101. For example, during delivery of IMD 102 to heart 101 using receptacle device 108, medical system 100 may be configured to utilize processing circuitry 134 mechanically supported by external device 136, a delivery system electrode of delivery system 120, and external electrode 138 to assess whether receptacle device 108 is within an atrium of heart 101, a ventricle of heart 101, a coronary sinus of heart 101, or some other portion of heart 101. Receptacle electrode 140 may conduct a signal from a fluid environment within heart 101 and substantially through receptacle wall 110 to reduce signal attenuation, shielding, and/or interference that might otherwise occur prior to the signal arriving at the delivery system electrode.

Additionally, during delivery of IMD 102 to heart 101, medical system 100 may be configured to utilize processing circuitry 134 mechanically supported by IMD 102 and one or more electrodes supported by IMD 102 to assess a proximity of IMD 102 (e.g., attachment member 132) to a vessel or chamber wall of heart 101, conduct pace mapping to determine a suitable placement of IMD 102, evaluate a suitability of pacing delivered by IMD 102 at a particular location, and/or for other reasons. Receptacle electrode 140 may conduct a signal from a fluid environment within heart 101 substantially through receptacle wall 110 to reduce signal attenuation, shielding, and/or interference that might otherwise occur prior to the signal arriving at the IMD return electrode. Further, receptacle electrode 140 may allow use of processing circuitry 134 mechanically supported by IMD 102 and electrodes supported by IMD 102 as IMD 102 resides at least partially within receptacle volume 112, such that repeated displacement of receptacle device 108 from IMD 102 within the relatively small confines of a heart chamber may be reduced and/or avoided.

In examples, medical system 100 is configured to use processing circuitry such as processing circuitry 131 when IMD 102 and processing circuitry 133 are substantially in an off state. For example, during delivery of IMD 102 to a chamber of the heart, medical system 100 may be configured to utilize delivery system electrode 158 (FIG. 3) and receptacle electrode 140 to sense a signal using processing circuitry 131. This may be used to, for example, determine a location of receptacle device 108 and IMD 102 during the IMD delivery. For example, medical system 100 may use processing circuitry 131, delivery system electrode 158, and receptacle electrode 140 to evaluate whether receptacle device 108 and IMD 102 are in an atrium or a ventricle of a patient. Medical system 100 may be configured to use processing circuitry such as processing circuitry 133 (FIG. 3) when IMD 102 and processing circuitry 133 are in a substantially on state. For example, during pace mapping and/or implantation of IMD 102 within a chamber of the heart, medical system 100 may be configured to utilize IMD distal electrode 162 (FIG. 3), IMD atrial electrode 164, IMD return electrode 166, and/or receptacle electrode 140 to sense a signal using processing circuitry 131. The use of processing circuitry 133 receiving signals substantially from electrodes of IMD 102, rather than the use of processing circuitry 131 receiving signals substantially from delivery system 120, may provide more precise signals during, for example, pace mapping, implantation of IMD 102, or other actions.

In examples, for example while utilizing processing circuitry 131 (FIG. 1), delivery system electrode 158 may be configured to electrically communicate with some portion of IMD 102 (e.g., IMD retrieval structure 148, IMD housing 160, and/or IMD return electrode 166 (FIG. 3)) to assist delivery system electrode 158 in receiving a signal for delivery to processing circuitry 131. For example, IMD retrieval structure 148 may include one or more conductive portions configured to contact delivery system electrode 158. IMD retrieval structure 148 may be in electrical communication with IMD housing 160 and/or IMD return electrode 166. Hence, in some examples, receptacle electrode 140 may be configured to conduct a signal from an environment surrounding an exterior of receptacle device 108 to one or more of IMD return electrode 166, IMD housing 160, IMD retrieval structure 148, and/or another portion of IMD 102, which may then individually or in conjunction communicate the signal to delivery system electrode 158 (e.g., even when IMD 102 and/or processing circuitry 133 are substantially in the off state). This may improve the reception of signals by delivery system electrode 158. In some examples, delivery system electrode 158 is configured to make physical contact with IMD retrieval structure 148 when delivery system 120 engages IMD retrieval structure 148, such that, for example, IMD retrieval structure 148 may communicate a signal to delivery system electrode 158 via the physical contact. In some examples, delivery system electrode 158 comprises some portion of a conductive surface of a head portion (e.g., head portion 128 (FIG. 3)) of delivery system 120. The conductive portion may be configured to make physical contact with retrieval structure 158 when head portion 128 engages retrieval structure 158.

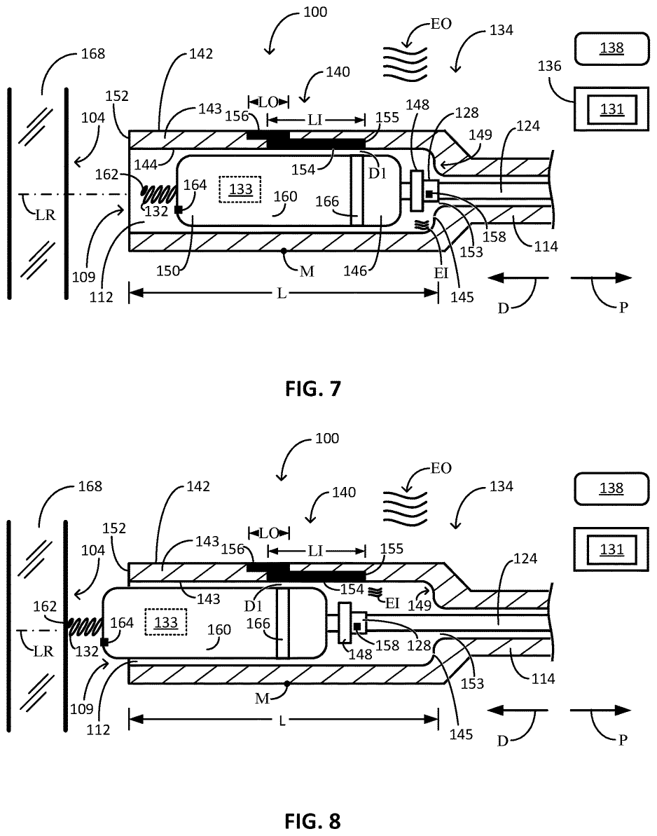

In some examples, medical system 100 (e.g., head portion 128) is configured to engage a proximal portion of IMD 102 (e.g., IMD proximal portion 146 (FIG. 3)) to transfer a torque to IMD 102. Delivery system body 122 may be configured to receive a torque (e.g., from a clinician) and transfer the torque to head portion 128. In examples, the IMD proximal portion includes a retrieval structure (e.g., IMD retrieval structure 148 (FIG. 7)). IMD retrieval structure 148 may be configured to engage with medical system 100 and/or another medical device to, for example, implant IMD 102 within an anatomical volume, retrieve IMD 102 from an anatomical volume, re-position IMD 102 within an anatomical volume, and/or re-orient IMD 102 within an anatomical volume. IMD 102 may include a distal portion 150 (“IMD distal portion 150”) opposite IMD proximal portion 146. In examples, IMD 102 (e.g., IMD distal portion 150) supports attachment member 132. In some examples, attachment member 132 is configured (e.g., as a helix) such that rotation of IMD 102 about a device axis LD defined by IMD 102 causes attachment member to engage and/or disengage tissues within target site 104.

For example, attachment member 132 may be configured such that rotation of IMD 102 in a first rotational direction W1 about device axis LD causes attachment member 132 to engage (or alternately, disengage from) tissues within target site 104. Attachment member 132 may be configured such that rotation of IMD 102 about device axis LD in a second rotational direction W2 substantially opposite first rotational direction W1 causes attachment member 132 to disengage from (or alternately, engage) tissues within target site 104. In examples, IMD 102 includes one or more components (e.g., a communication antenna, a sensor, or another component) configured to rotate around and or revolve about device axis LD when IMD 102 rotates about device axis LD. Medical system 100 may cause IMD 102 to rotate about device axis LD to cause one or more of the components to substantially establish a specific orientation with respect to the anatomy of the patient, another device implanted within or worn by the patient, another device external to the patient, and/or other devices. Medical system 100 may cause rotation of IMD 102 in the first rotational direction W1 and/or the second rotation direction W2 to, for example, implant IMD 102, retrieve IMD 102, re-position IMD 102, and/or re-orient IMD 102 within an anatomical volume such as the RA. In some examples, medical system 100 may include a snare configured to engage IMD 102 (e.g., IMD retrieval structure 148).

Although the examples herein discuss delivery, retrieval, and/or positioning of IMD 102 within the RA of heart 101, medical system 100 may be configured to deliver, retrieve, and/or position IMD 102 in any of the other chambers of heart 101 and/or in other anatomical volumes of a patient in a like manner as that described for the RA of heart 101. Further, although the examples herein discuss attachment member 132 defining a helix, attachment member 132 may defines other structures, such as one or more elongated tines extending from, for example, IMD distal portion 150. Further, delivery system 120 may be configured to exert a translational force (e.g., in the distal direction D) on IMD 102 to cause attachment member 132 to engage tissues. The translational force exerted by delivery system 120 may cause IMD 102 to move in the distal direction D relative to receptacle device 108 such that, for example, attachment member 132 engages the tissues. Target site 104 may include an appendage of the RA, or the triangle of Koch region of the RA, or some other portion of heart 101, or some other location within a body of a patient.

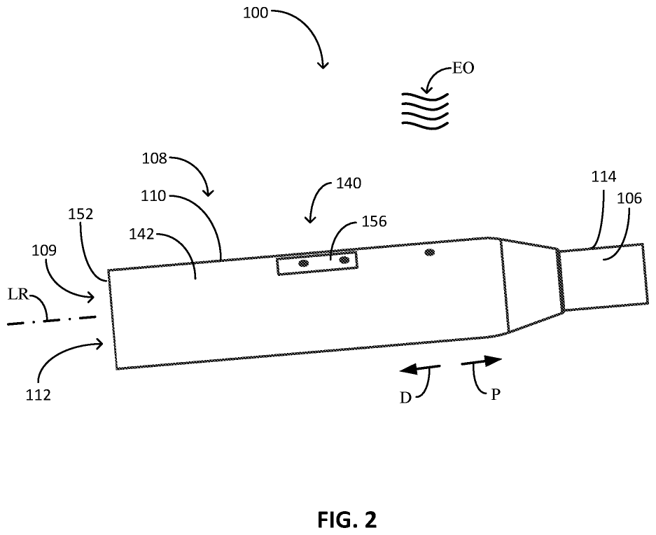

FIG. 2 is a perspective view of a portion of medical system 100 including receptacle device 108, with receptacle device 108 including receptacle electrode 140. FIG. 3 is a perspective view of a portion of medical system 100 including IMD 102 positioned within receptacle volume 112 defined by receptacle wall 110, with receptacle wall 110 illustrated as transparent in FIG. 3 for clarity. FIG. 4 is a cross-sectional plan view of receptacle device 108, with the cutting plane taken parallel to the page.

Receptacle device 108 includes receptacle wall 110. Receptacle wall 110 includes a body 143 (“wall body 143”) (FIG. 4) defining wall inner surface 144. Wall inner surface 144 may define at least a portion of a boundary defining receptacle volume 112. In examples, wall inner surface 144 at least partially and/or substantially completely surrounds a receptacle axis LR defined by receptacle device 108. Wall inner surface 144 may be configured such that wall inner surface 144 substantially faces in a direction substantially toward receptacle axis LR. For example, at least some portion of wall inner surface 144 may be configured such that a vector V1 normal to and extending away from wall inner surface 144 extends in a direction from wall inner surface 144 toward receptacle axis LR. In some examples, vector V1 is substantially perpendicular to receptacle axis LR.

In examples, receptacle device 108 (e.g., wall body 143) defines receptacle opening 109 which opens into receptacle volume 112. Receptacle device 108 may define receptacle opening substantially at a distal end 152 of receptacle device 108 (“receptacle distal end 152”). In examples, wall body 143 defines receptacle distal end 152.

Receptacle opening 109 may be configured such that IMD 102 may egress from within receptacle volume 112 through receptacle opening 109 and/or ingress into receptacle volume 112 through receptacle opening 112. In examples, receptacle axis LR extends through at least some portion of receptacle volume 112 and extends through receptacle opening 109. In examples, receptacle volume 112 is bounded at least partially by receptacle opening 112.

In examples, delivery catheter 106 (e.g., delivery catheter distal portion 114) defines a lumen opening 153 of delivery lumen 118 (“delivery lumen opening 153”) which opens into receptacle volume 112. Delivery lumen opening 153 is configured to allow at least some portion of delivery system body 122 to position within receptacle volume 112 when another portion of delivery system body 122 is positioned within delivery lumen 118. In examples, receptacle axis LR extends through delivery lumen opening 153. In some examples, receptacle axis LR extends through delivery lumen opening 153, receptacle opening 109, and at least some portion of receptacle volume 112. Receptacle volume 112 may be bounded at least partially by delivery lumen opening 153. In examples, receptacle volume 112 is bounded at least in part by delivery lumen opening 153, receptacle opening 109, and at least some portion of wall inner surface 144. In some examples, receptacle volume 112 defines a cross-sectional area perpendicular to receptacle axis LR, with the cross-sectional area defined by a curved, curvilinear, or polygonal boundary. In some examples, the cross-sectional area is defined by a substantially circular boundary.

Wall body 143 may define wall outer surface 142. In examples, at least some portion of wall body 143 is between wall outer surface 142 and wall inner surface 144. In examples, wall outer surface 142 at least partially and/or substantially completely surrounds receptacle axis LR and/or wall inner surface 144. Wall outer surface 142 may be configured such that wall outer surface 142 substantially faces in a direction away from receptacle axis LR, wall inner surface 144, and/or receptacle volume 112. For example, at least some portion of wall outer surface 142 may be configured such that a vector V2 normal to and extending away from wall outer surface 142 extends in a direction from wall outer surface 142 away from receptacle axis LR, wall inner surface 144, and/or receptacle volume 112. In some examples, vector V2 is substantially perpendicular to receptacle axis LR. In some examples, vector V2 is substantially parallel to vector V1.

Wall outer surface 142 is configured to be in fluidic communication with an external environment EO surrounding an exterior of receptacle device 108. For example, wall outer surface 142 may be configured to be in fluid communication with a fluid environment within a chamber of heart 101 when receptacle device 108 is positioned (e.g., by a clinician) within heart 101. In examples, external environment EO comprises a fluid (e.g., blood of a patient), and receptacle device 108 is configured to establish an internal environment EI comprising the fluid or another fluid within receptacle volume 112 when wall outer surface fluidically communicates with external environment EO. Receptacle device 108 may be configured such that the external environment EO and the internal environment EI may exchange one or more fluids via receptacle opening 109.

Receptacle electrode 140 is configured to define a conductive path through receptacle wall 110, such that a signal (e.g., an intrinsic cardiac signal of heart 101) may travel through the conductive path from external environment EO to receptacle volume 112 (e.g., to internal environment EI within receptacle volume 112). In examples, receptacle electrode 140 extends through receptacle wall 110. In examples, receptacle electrode 140 extends from wall outer surface 142 to wall inner surface 144. Receptacle electrode 140 may be substantially supported by and/or embedded within receptacle wall 110. In examples, receptacle wall 110 comprises a first material (e.g., a low conductivity polymer) having a first electrical conductivity, and receptacle electrode 140 comprises a second material (e.g., a metal and/or conductive polymer) having a second electrical conductivity greater than the first electrical conductivity, such that receptacle electrode 140 defines the conductive path through receptacle wall 110. In examples, receptacle electrode 140 is configured such that a signal traveling from external environment EO to internal environment EI preferentially follows a signal path through receptacle electrode 140 over a signal path through receptacle wall 110.

Receptacle electrode 140 may define an inner electrode face 154 facing substantially toward receptacle volume 112 (and, e.g., internal environment EI) and an outer electrode face 156 (FIG. 2) facing substantially away from receptacle volume 112 (e.g., facing the external environment EO). In an example, receptacle electrode 140 includes a body 155 (“receptacle electrode body 155”) defining inner electrode face 154 and/or outer electrode face 156. Receptacle electrode 140 may be configured to receive a signal (e.g., an intrinsic cardiac electrical signal) via outer electrode face 156 and communicate the signal to receptacle volume 112 using inner electrode face 154. Receptacle volume 112 may be configured to communicate the signal to an electrode positioned within receptacle volume 112 (e.g., an electrode of IMD 102 and/or an electrode of delivery system 120) using, for example, a fluid comprising internal environment EI within receptacle volume 112. In some examples, receptacle electrode 140 includes one or more conductors supported by receptacle wall 110 and configured to electrically connect an outer electrode body defining outer electrode face 156 and an inner electrode body defining inner electrode face 154.

For example, as illustrated in FIG. 3, delivery system 120 (e.g., delivery system body 122 and/or head portion 128) may include a delivery system electrode 158. Delivery system electrode 158 may be mechanically supported by delivery system body 122 and/or head portion 128. In examples, delivery system 120 is configured such that delivery system electrode 158 may communicate with processing circuitry 134 (e.g., device processing circuitry 131). When delivery system electrode 158 is positioned within receptacle volume 112, receptacle wall 110 and/or a housing 160 of IMD 102 (“IMD housing 160”) may act to substantially attenuate, shield, and/or otherwise interfere with a signal as the signal travels over a signal path from external environment EO to delivery system electrode 158 (e.g., a signal path through receptacle opening 109).

Receptacle electrode 140 may be configured to define a signal path substantially through receptacle wall 110, such that attenuation, shielding, and/or interference of the signal along a signal path from external environment EO to delivery system electrode 158 may be mitigated and/or reduced. In examples, receptacle electrode 140 is configured to receive the signal via outer electrode face 156 and substantially emit the signal into reception volume 112 via inner electrode face 154. In examples, receptacle electrode 140 is configured to emit the signal to internal environment EI. Receptacle device 108 and/or delivery system 120 may be configured such that delivery system electrode 158 is in fluidic communication with internal environment EI, and such that internal environment EI may provide a path for the signal from inner electrode face 154 to delivery system electrode 158. Delivery system electrode 158 may subsequently provide the signal to processing circuitry 134 (e.g., device processing circuitry 131). Processing circuitry 134 may process and/or condition the signal thus sensed by medical system 100. For example, delivery system electrode 158 may provide the signal such that processing circuitry 134 (e.g., device processing circuitry 131) may assess a location of receptacle device 108, such as whether receptacle device 108 is within an atrium of heart 101, a ventricle of heart 101, a coronary sinus of heart 101, or some other portion of heart 101.

In some examples, IMD 102 mechanically supports one or more device electrodes 161 (“device electrodes 161”), such as an IMD distal electrode 162 supported by attachment member 132, IMD atrial electrode 164 supported by IMD distal portion 150, an IMD return electrode 166 supported by IMD housing 160, and/or other electrodes supported by IMD 102. Device electrodes 161 may be configured to communicate with processing circuitry 134 (e.g., IMD processing circuitry 133). Processing circuitry 134 may be configured to process and/or condition a signal sensed by device electrodes 161 and/or other electrodes within medical system 100. In examples, at least one of device electrodes 161 (e.g., IMD return electrode 166) is configured to be positioned with receptacle volume 112 when IMD 102 resides at least partially within receptacle volume 112. In examples, IMD 102 is configured such that at least one of electrodes 161 (e.g., at least one of IMD distal electrode 162, IMD atrial electrode 164, and/or IMD return electrode 166) is in fluidic communication with internal environment EI when IMD 102 resides at least partially within receptacle volume 112.

When at least one of device electrodes 161 (e.g., IMD return electrode 166) is positioned within receptacle volume 112, and in a manner similar to that described for delivery system electrode 158, receptacle wall 110 and/or IMD housing 160 may act to substantially attenuate, shield, and/or interfere with a signal traveling over a signal path (e.g., through receptacle opening 109) from external environment EO to the at least one of device electrodes 161 (e.g., IMD return electrode 166). Receptacle electrode 140 may be configured to receive the signal via outer electrode face 156 and substantially emit the signal into reception volume 112 via inner electrode face 154, such that internal environment EI may provide a path for the signal from inner electrode face 154 to device electrodes 161 (e.g., IMD return electrode 166). Device electrodes 161 may subsequently provide the signal to processing circuitry 134 (e.g., IMD processing circuitry 133) such that processing circuitry 134 may process and/or condition the signal thus sensed by medical system 100. For example, device electrodes 161 may provide the signal such that processing circuitry 134 (e.g., IMD processing circuitry 133) may assess electrical activity in proximity of IMD 102 (e.g., attachment member 132) to a vessel wall of heart 101, conduct pace mapping to determine a suitable placement of IMD 102, evaluate suitability of pacing delivered by IMD 102 at a particular location, and/or for other reasons. Further, allowing IMD 102 to be substantially “on” may allow use of processing circuitry 131 to determine when IMD 102 may be in a proper position during an implantation. For example, processing circuitry 131 may be utilized during an implantation to determine when sufficient rotations of IMD 102 have occurred (e.g., using delivery system 120) such that, for example, IMD distal electrode 162 and/or IMD atrial electrode 164 are in proper position within a vessel wall. Hence, receptacle electrode 140 may allow use of IMD processing circuitry 133 and device electrodes 161 mechanically supported by IMD 102 as IMD 102 resides at least partially within receptacle volume 112, such that repeated displacements of receptacle device 108 from IMD 102 within the relatively small confines of a heart chamber may be reduced and/or avoided.

Delivery system body 122 may extend through delivery lumen 118 of delivery catheter 106. In examples, delivery system body 122 mechanically supports head portion 128. Delivery system body 122 and delivery lumen 118 are shown in dashed lines in FIG. 3. Head portion 128 is configured to engage IMD 102 (e.g., IMD proximal portion 146) within receptacle volume 112. In examples, head portion 128 is configured to engage a retrieval structure 148 of IMD proximal portion 146. Delivery system body 122 and/or head portion 128 are configured to translate within delivery lumen 118 in the distal direction D and/or proximal direction P relative to delivery catheter 106 and receptacle device 108. In examples, delivery system body 122 and/or head portion 128 are configured to cause translation of IMD 102 in the proximal direction P and/or distal direction D relative to receptacle device 108 when delivery system 120 (e.g., head portion 128) engages IMD proximal portion 146 and delivery system body 122 and/or head portion 128 translate in the proximal direction P and/or distal direction D relative to delivery catheter 106 and receptacle device 108. Delivery system 120 may be configured such that a force (e.g., in the proximal direction P and/or the distal direction D) exerted on delivery system body 122 (e.g., by a clinician) causes head portion 128 to transfer the force to IMD 102 when delivery system 120 (e.g., head portion 128) engages IMD 102.

Delivery system body 122 and head portion 128 may be configured to rotate (e.g., within delivery lumen 118) about a delivery system axis LB defined by delivery system body 122. Delivery system body 122 and head portion 128 may rotate about delivery system axis LB relative to delivery catheter 106 and receptacle device 108. Delivery system body 122 and/or head portion 128 may be configured to cause rotation of IMD 102 about device axis LD relative to receptacle device 108 when delivery system 120 (e.g., head portion 128) engages IMD proximal portion 146 and delivery system body 122 and/or head portion 128 rotate about delivery system axis LB relative to delivery catheter 106 and receptacle device 108. Delivery system 120 may be configured such that a torque imparted on delivery system body 122 (e.g., by a clinician) causes delivery system 120 to transfer the torque to IMD 102 when delivery system 120 (e.g., head portion 128) engages IMD 102.

In examples, delivery catheter 106 is configured such that delivery system body distal portion 124 may slidably translate through delivery lumen opening 153 via delivery lumen 118, such that portions of delivery system body distal portion 124 may be positioned (e.g., by a clinician) within both delivery lumen 118 and receptacle volume 112. Delivery catheter 106 and/or receptacle device 108 may be configured such that head portion 128 positions and/or translates (e.g., in the distal direction D and/or proximal direction P) within receptacle volume 112 and/or delivery lumen 118 when delivery system body distal portion 124 positions and/or translates within delivery lumen 118 and/or receptacle volume 112. In examples, delivery system 102 is configured to allow removal of delivery system 120 (e.g., by withdrawing proximally) from delivery catheter 106 to, for example, insert another device within delivery lumen 118.

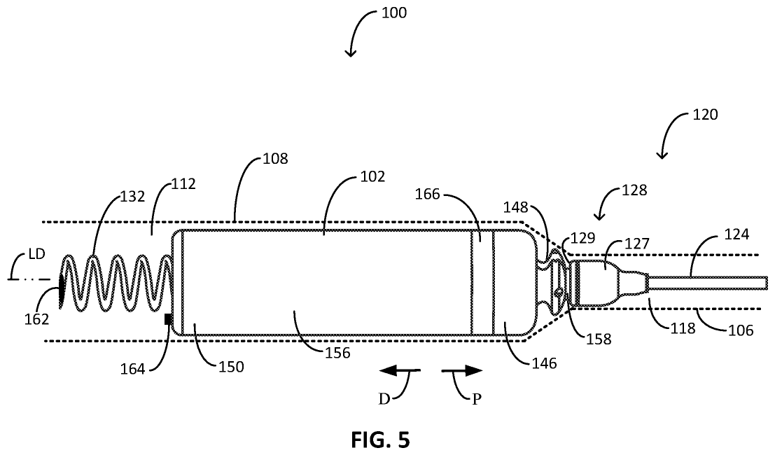

For example, FIG. 5 illustrates a plan view of IMD 102 (e.g., IMD retrieval structure 148) engaged by head portion 128 of delivery system 120. Head portion 128 is coupled to delivery system body distal portion 124. Delivery catheter 106 and receptacle device 108 are represented schematically with dashed lines in FIG. 5. Delivery system 120 is configured such that head portion 128 may translate (e.g., relative to receptacle device 108) in the distal direction D from delivery lumen 118 to into receptacle volume 112 when, for example, a force in the distal direction D is imparted on head portion 128 by delivery system body distal portion 124 (e.g., caused by a physician). Delivery system 120 is configured such that head portion 128 may translate in the proximal direction P (e.g., relative to receptacle device 108) from receptacle volume 112 into delivery lumen 118 when, for example, a force in the proximal direction P is imparted on head portion 128 by delivery system body distal portion 124 (e.g., caused by a physician).

Head portion 128 is configured to cause translation of IMD 102 relative to receptacle device 108 when head portion 128 engages IMD 102 (e.g., IMD retrieval structure 148) and delivery system body distal portion 124 exerts a force on head portion 128. For example, head portion 128 may be configured to cause IMD 102 to translate in the proximal direction P (e.g., relative to receptacle device 108) when head portion 128 engages IMD 102 (e.g., IMD retrieval structure 148) and delivery system body distal portion 124 exerts a force in the proximal direction P. Head portion 128 may be configured to cause IMD 102 to translate in the distal direction D when head portion 128 engages IMD 102 (e.g., IMD retrieval structure 148) and delivery system body distal portion 124 exerts a force in the distal direction D. In examples, head portion 128 is configured to cause rotation of IMD 102 relative to receptacle device 108 when head portion 128 engages IMD 102 and delivery system body distal portion 124 exerts a torque on head portion 128. For example, head portion 128 may be configured to cause IMD 102 to rotate in the first rotational direction W1 (FIG. 1) when head portion 128 engages IMD 102 (e.g., IMD retrieval structure 148) and delivery system body distal portion 124 exerts a torque on head portion 128 (e.g., around device axis LD) in the first rotational direction W1. Head portion 128 may be configured to cause IMD 102 to rotate in the second rotational direction W2 (FIG. 1) when head portion 128 engages IMD 102 (e.g., IMD retrieval structure 148) and delivery system body distal portion 124 exerts a torque on head portion 128 (e.g., around device axis LD) in the second rotational direction W2.

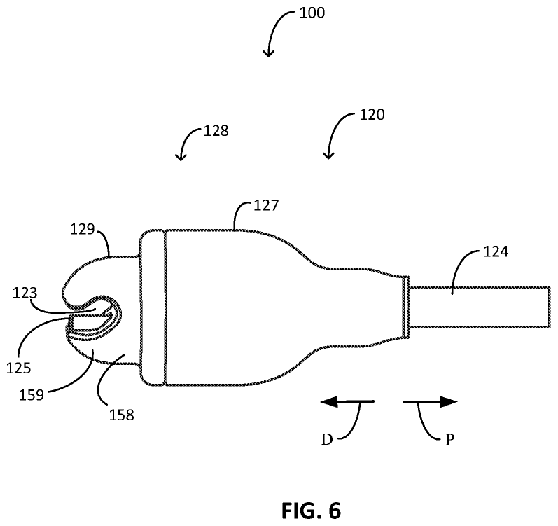

FIG. 6 illustrates an example head portion 128 coupled to delivery system body distal portion 124. Head portion 128 includes a support section 127 supporting an engagement section 129. Support section 127 may be configured to couple to and/or coupled to delivery system body distal portion 124. Engagement section 129 may be configured to engage IMD 102 (e.g., IMD retrieval structure 148). In examples, engagement section 129 is configured to disengage from IMD 102. For example, head portion 128 may include an engagement member 125 configured to move relative to engagement section 129 to cause head portion 128 to engage with and/or disengage from IMD 102. For example, engagement section 129 may define an aperture 123 configured to receive IMD retrieval structure 148 when head portion 128 engages IMD 102. Engagement member 125 may be configured to move relative to engagement section 129 (e.g., move in the distal direction D) to reduce a size of aperture 123, such that IMD retrieval structure 148 remains substantially trapped within aperture 123. Head portion 128 may be configured to exert a distally directed force, proximally directed force, or torque around device axis LD when IMD retrieval structure 148 is trapped within aperture 123. Engagement member 125 may be configured to move relative to engagement section 129 (e.g., move in the proximal direction P) to increase a size of aperture 123, such that head portion 128 may disengage from retrieval structure 148 (e.g., IMD retrieval structure 148 may be released from aperture 123). For example, engagement member 125 may move relative to engagement section 129 such that proximal translation of head portion 128 relative to IMD 102 causes head portion 128 to disengage from IMD retrieval structure 148 (e.g., following implantation of IMD 102 in a patient using delivery system 120).

As discussed, delivery system 120 may include delivery system electrode 158. Delivery system electrode 158 may be mechanically supported by delivery system body 122 and/or head portion 128. In examples, engagement section 129 supports delivery system electrode 158. In some examples, delivery system electrode 158 comprises some portion of or substantially all of housing surface 159 of a housing of engagement section 129. For example, housing surface 159 may be a conductive material configured to be covered by an insulative material. Delivery system electrode 158 may some portion of housing surface 159 having the insulative cover removed, such that delivery system electrode 158 may receive a signal (e.g., from internal environment EI) and enable communication of the signal to processing circuitry 134 (e.g., processing circuitry 131).

In some examples, housing surface 159 may be substantially entirely uncovered by any insulative material, such that delivery system electrode 158 comprises substantially the entirety of housing surface 159. In examples, housing surface 159 is configured to be fluidic communication with receptacle volume 112 and/or delivery lumen 118 when head portion 128 resides within receptacle volume 112 and/or delivery lumen 118.

In examples, receptacle device 108 is configured to substantially prevent (e.g., mechanically prevent) IMD 102 from freely passing (e.g., completely passing) through delivery lumen opening 153 into delivery lumen 118. Delivery catheter 106 and/or receptacle device 108 may be configured to substantially trap at least a portion of IMD 102 within receptacle volume 112. In examples, medical system 100 (e.g., receptacle device 108 and/or delivery catheter 106) includes a proximal stop 149 (FIGS. 4, 7, 8) configured to limit movement of IMD 102 within receptacle volume 112 when IMD 102 translates in the proximal direction P relative to receptacle wall 110. In some examples, proximal stop is configured to substantially prevent (e.g., mechanically prevent) head portion 128 from freely passing (e.g., completely passing) through delivery lumen opening 153 into delivery lumen 118.

In examples, proximal stop 149 is configured to contact IMD 102 and/or head portion 128 when IMD 102 and/or head portion 128 translates in the proximal direction P relative to receptacle wall 110. In examples, when IMD 102 and/or head portion 128 contacts and exerts a force in the proximal direction P on proximal stop 149, proximal stop 149 may be configured to exert a reaction force on IMD 102 and/or head portion 128 in the distal direction D. Proximal stop 149 may be configured such that the reaction force limits and/or ceases movement of IMD 102 and/or head portion 128 in the proximal direction P, such that at least a portion of IMD 102 and/or head portion 128 is substantially prevented from entering delivery lumen 118 via delivery lumen opening 153.

In examples, delivery lumen 118 defines a first radius R1 extending from receptacle axis LR and/or body axis LB. Delivery lumen 118 may be configured such that first radius R1 substantially prevents at least some portion of IMD 102 and/or head portion 128 from passing into delivery lumen 118 via delivery lumen opening 153. An inner wall 147 of delivery catheter 106 (“delivery catheter inner wall 147”) may define first radius R1. Receptacle volume 112 may define a second radius R2 extending from receptacle axis LR and greater than first radius R1. Wall inner surface 144 may define second radius R2. In examples, receptacle device 108 defines a proximal wall 145. Proximal wall 145 may be configured to transition from wall inner surface 144 to delivery catheter inner wall 147. In some examples, proximal wall 145 is configured to transition from the second radius R2 to the second radius R1.

In some examples proximal wall 145 is defined wholly by receptacle device 108 or delivery catheter distal portion 114. In some examples, receptacle device 108 defines a first portion of proximal wall 145 and delivery catheter distal portion 114 defines a second portion of proximal wall 145. Proximal wall 145 may define proximal stop 149. For example, in some examples, proximal stop 149 is a surface defined by and/or extending from proximal wall 145. In other examples, proximal stop 149 may be surface defined by and/or extending from wall inner surface 144, delivery catheter inner wall 147, or another portion of receptacle device 108 and/or delivery catheter 106.

In examples, receptacle electrode 140 may be configured such that inner electrode face 154 and at least one of device electrodes 161 (e.g., IMD return electrode 166) remain in relative proximity to one another as IMD 102 is translated (e.g., by a clinician using, e.g., delivery system 120) within receptacle volume 112. Receptacle electrode 140 may be configured to substantially maintain and/or establish connectivity (e.g., either by contact or via internal environment EI) with the at least one of device electrodes 161 as IMD 102 is translated in the distal direction D and/or the proximal direction P to different positions within receptacle volume 112. For example, receptacle device 108 may be configured to hold IMD 102 in a delivery position within receptacle volume 112 as medical system 100 transports receptacle device 108 and the IMD 102 (e.g., via vasculature) to a target site (e.g., target site 104 (FIG. 1)). Once in the vicinity of the target site, medical system 100 (e.g., under the influence of clinician) may be configured to translate IMD 102 within receptacle volume 112 in the distal direction D, toward receptacle opening 109. Inner electrode face 154 may be configured to substantially maintain a proximity with at least one of device electrodes 161 (e.g., IMD return electrode 166) as the IMD 102 is translated in the distal direction D and/or the proximal direction P within receptacle volume 112. In some examples, inner electrode face 154 is configured to substantially maintain and/or establish connectivity with the at least one of device electrodes 161 (e.g., IMD return electrode 166) as the IMD 102 is translated in the distal direction D and/or the proximal direction P within receptacle volume 112.

For example, FIG. 7 illustrates a cross-sectional schematic illustration of IMD 102 positioned within receptacle volume 112 of receptacle device 108, with IMD 102 in proximity to a vessel wall 168 of heart 101. In FIG. 7, IMD 102 is in a first position within receptacle volume 112. In examples, the first position is a delivery position of IMD 102 within receptacle volume 112. Receptacle device 108 may be configured such that at least IMD distal portion 150 and/or IMD atrial electrode 164 is proximal to receptacle opening 109 in the first position (e.g., the delivery position). In examples, attachment member 132 and/or IMD distal electrode 162 is proximal to receptacle opening 109 in the first position (e.g., the delivery position). FIG. 8 illustrates a cross-sectional schematic illustration of IMD 102 positioned within receptacle volume 112 in a second position distal to the first position. Receptacle device 108 may be configured such that at least some portion of attachment member 132 and/or IMD distal portion 150 is distal to receptacle opening 109 in the second position. In examples, in the second position, at least some portion of IMD distal portion 150 is distal to receptacle opening 109 and at least some portion of IMD proximal portion 146 is proximal to receptacle opening 109. In some examples, in the second position, IMD distal electrode 162 and/or IMD atrial electrode 164 is distal to receptacle opening 109 and IMD return electrode 166 is proximal to receptacle opening 109. The cross-sections of FIG. 7 and FIG. 8 are illustrated with a cutting plane taken parallel to the page. In examples, medical system 100 is configured to conduct pace mapping using one or more of IMD distal electrode 162, receptacle electrode 140, IMD return electrode 166, delivery system electrode 158, and/or other electrodes of medical system 100. For example, medical system 100 may be configured to conduct pace mapping when IMD distal electrode 162 is in contact with and/or close proximity to vessel wall 168.

In some of the examples of this disclosure, the configuration of receptacle electrode 140 may be described with respect to IMD return electrode 166 and/or delivery system electrode 158 for ease of description, however the descriptions may apply to other electrodes of medical system 100 in other examples.

In examples, receptacle device 108 is configured to substantially establish and/or maintain a proximity to IMD return electrode 166 through contact (e.g., mechanical contact) between inner electrode face 154 and IMD return electrode 166 when IMD 102 resides within receptacle volume 112. For example, receptacle device 108 may be configured such that inner electrode face 154 and IMD return electrode 166 substantially maintain a sliding contact when IMD 102 resides within receptacle volume. Inner electrode face 154 may be configured to substantially maintain the sliding contact when IMD 102 translates (e.g., is translated by delivery system 120) in the proximal direction P and/or the distal direction D within receptacle volume 112 and relative to receptacle wall 110 (e.g., when IMD 102 is positioned in the first position or the second position, and/or when IMD 102 transitions between the first position and the second position). In some examples, inner electrode face 154 is configured to substantially maintain the sliding contact when IMD 102 rotates (e.g., is rotated by delivery system 120) about device axis LD and/or receptacle axis LR within receptacle volume 112 and relative to receptacle wall 110.

In some examples, receptacle device 108 is configured to substantially establish and/or maintain a proximity to IMD return electrode 166 by substantially establishing and/or maintaining a displacement between inner electrode face 154 and IMD return electrode 166 when IMD 102 resides within receptacle volume 112. The displacement may reduce and/or eliminate signal noise and/or other signal perturbations that might result as IMD 102 translates and/or rotates within receptacle volume 112. For example, the translation and/or rotation of IMD 102 may cause intermittent contact between inner electrode face 154 and the one or more of device electrodes 161 (e.g., IMD return electrode 166), potentially introducing signal noise of other disturbance of the signal as the signal travels (e.g., via internal environment EI) from inner electrode face 154 to IMD return electrode 166, and/or travels to another electrode of medical system 100, such as delivery system electrode 158. In examples, receptacle device 108 is configured to maintain a displacement D1 between inner electrode face 154 and IMD return electrode 166 when IMD 102 translates (e.g., is translated by delivery system 120) in the proximal direction P and/or the distal direction D within receptacle volume 112 and relative to receptacle wall 110 (e.g., when IMD 102 is positioned in the first position or the second position, and/or when IMD 102 transitions between the first position and the second position). In examples, the displacement DI is substantially perpendicular to receptacle axis LR.

Inner electrode face 154 may be configured to extend in the proximal direction P and/or the distal direction D within the receptacle volume 112, such that, for example, receptacle electrode 140 remains in relative proximity with IMD return electrode 166 when IMD 102 is positioned in the first position or the second position, and/or when IMD 102 transitions between the first position and the second position. In examples, receptacle device 108 defines a length L from receptacle distal end 152 to proximal wall 145. Length L may be substantially parallel to receptacle axis LR, device axis LD, and/or some portion of body axis LB. Receptacle electrode 140 may be configured to provide the sliding contact with IMD return electrode 166 and/or the displacement D1 from IMD return electrode 166 as IMD 102 translates over some portion of the length L. In examples, inner electrode face 154 defines an inner electrode length LI substantially parallel to length L. In some examples, inner electrode length LI is at least 10% of length L, in some examples at least 20% of length L, in some examples at least 35% of length L, and in some examples at least 50% of length L. Hence, receptacle electrode 140 may be configured such that inner electrode face 154 provides the sliding contact with IMD return electrode 166 and/or the displacement D1 from IMD return electrode 166 when IMD 102 is in the first position within receptacle volume 112 (FIG. 7) and in the second position within receptacle volume 112 (FIG. 8), and/or when IMD 102 is in a position within receptacle volume 112 between the first position and the second position.

In examples, outer electrode face 156 defines an outer electrode length LO substantially parallel to length L and/or inner electrode length LI. In examples, outer electrode length LO is less than or equal to inner electrode length LL Outer electrode length LO may be less than inner electrode length LI to, for example, minimize impacts on the structural strength and/or flexibility of receptacle wall 110. In some examples, outer electrode length LO is greater than inner electrode length LI. In examples, outer electrode length LO is at least 10% of length L, in some examples at least 20% of length L, in some examples at least 35% of length L, and in some examples at least 50% of length L.

As discussed, medical system 100 may be configured to communicate a signal (e.g., from external environment EO to internal environment EI) such that processing circuitry 134 may process and/or condition the sensed signal. Receptacle electrode 140 may be configured to reduce attenuation, shielding, and/or interference as the signal travels over a signal path from external environment EO to one or more of electrodes 161, delivery system electrode 158, and/or other electrodes of medical system 100. In examples, receptacle electrode 140 is configured to improve and/or enable more effective use of device processing circuitry 131 and/or IMD processing circuitry 133 during deployment, retrieval, and/or repositioning of IMD 102 using receptacle device 108.

For example, with IMD 102 within receptacle volume 112 (e.g., in the first position of FIG. 7), receptacle electrode 140 may be configured to provide a conductive path through receptacle wall 110 to reduce attenuation, shielding, and/or interference as a signal travels over a signal path from external environment EO to delivery system electrode 158. Delivery system electrode 158 may communicate the signal to device processing circuitry 131. Device processing circuitry 131 may be mechanically supported by external device 136 extracorporeal to the patient and/or otherwise displaced from receptacle device 108 and/or IMD 102. Medical system 100 may be configured to use device processing circuitry 131, delivery system electrode 158, and another electrode such as external electrode 138 to assess whether receptacle device 108 is within an atrium of heart 101, a ventricle of heart 101, a coronary sinus of heart 101, or some other portion of heart 101. Hence, receptacle electrode 140 may assist medical system 100 in receiving the signal from external environment EO when device processing circuitry 131 relies primarily on delivery system electrode 158 and external electrode 138. For example, receptacle electrode 140 may assist medical system 100 in receiving the signal when IMD 102 is in the first position within receptacle volume 112 (e.g., as depicted in FIG. 7).

As another example, for example with IMD 102 at least partially extending distal to receptacle opening 109 (e.g., in the second position of FIG. 8), receptacle electrode 140 may be configured to provide a conductive path through receptacle wall 110 to reduce attenuation, shielding, and/or interference as a signal travels over a signal path from external environment EO to IMD return electrode 166. IMD return electrode 166 may communicate the signal to IMD processing circuitry 133 mechanically supported by IMD housing 160. Medical system 100 may be configured to use IMD processing circuitry 133, IMD return electrode 166, and another electrode such as IMD distal electrode 162, IMD atrial electrode 164, and/or another of electrodes 161 to assess a proximity of IMD 102 (e.g., attachment member 132) to a vessel or chamber wall of heart 101, conduct pace mapping to determine a suitable placement of IMD 102, evaluate a suitability of pacing delivered by IMD 102 at a particular location, and/or for other reasons. For example, receptacle electrode 140 may assist medical system 100 in receiving the signal when IMD 102 is in the second position within receptacle volume 112 (e.g., as depicted in FIG. 8).

In some examples, receptacle electrode 140 is configured such that outer electrode face 156 is disposed within a distal wall portion of receptacle wall 110. Positioning outer electrode face 156 within the distal wall portion may assist in determining a location of receptacle distal end 152 within or relative to heart 101 using processing circuitry 134. In examples, receptacle wall 110 defines a midpoint M substantially halfway between delivery lumen opening 153 and receptacle opening 109. The midpoint M may be displaced proximally from receptacle distal end 152 and/or receptacle opening 109 by 50% of the length L defined by receptacle device 108, with the distal wall portion of receptacle wall 110 extending distal to the midpoint M. In examples, outer electrode face 156 is configured such that at least some portion of outer electrode face 156 extends into the distal wall potion. In some examples, a majority (e.g., greater than 50%) of outer electrode face 156 extends into the distal wall portion. In some examples, the entirety of outer electrode face 156 is surrounded by the distal wall portion. In some examples, the outer electrode face is extends to or is substantially adjacent receptacle distal end 152 and/or receptacle opening 109.

Hence, receptacle electrode 140 may improve the use of IMD processing circuitry 133 as IMD 102 resides at least partially within receptacle volume 112. Receptacle electrode 140 may be configured to substantially establish and/or maintain a proximity with IMD return electrode 166 as IMD 102 is translated (e.g., using delivery system 120, by a clinician) in the distal direction D and/or the proximal direction P during, for example, pace mapping using IMD distal electrode 162 and/or evaluation of pacing delivered by IMD 102. Thus, receptacle electrode 140, by providing a conductive signal path through receptacle wall 110, may improve the use of IMD processing circuitry 133 such that repeated displacement of receptacle device 108 from IMD 102 (e.g., while utilizing IMD processing circuitry 133) within the relatively small confines of a heart chamber of heart 101 may be reduced and/or avoided. Minimizing displacements of receptacle device 108 from IMD 102 may reduce a need to recapture IMD 102 within receptacle device 108 within the heart chamber to, for example, relocate IMD 102 within the heart chamber.

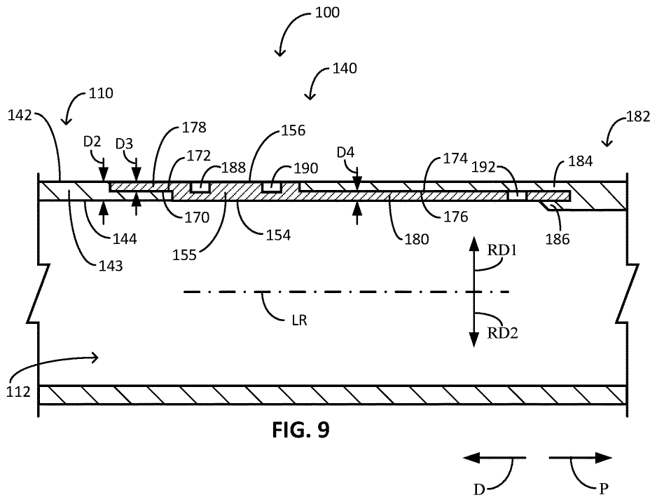

FIG. 9 is a cross-sectional schematic illustration of a portion of receptacle device 108 including receptacle electrode 140, with the cutting plane taken parallel to the page. In examples, receptacle device 108 is configured to limit and/or constrain movement of receptacle electrode 140 with respect to wall 110. For example, receptacle device 108 may be configured to limit and/or constrain movement of receptacle electrode 140 with respect to wall 110 in the distal direction D and/or the proximal direction P. Receptacle device 108 may be configured to limit and/or constrain movement of receptacle electrode 140 with respect to wall 110 in a first radial direction RD1 of receptacle device 108 (e.g., a radial direction substantially perpendicular to receptacle axis LR) and/or a second radial direction RD2 opposite first radial direction RD1.

In examples, wall body 143 defines an outer bearing surface 170 configured to limit and/or constrain movement of receptacle electrode 140 with respect to wall 110 in a direction toward receptacle axis LR (e.g., in the second radial direction RD2). Outer bearing surface 170 may be configured to contact and/or be in contact with receptacle electrode 140 (e.g., receptacle electrode body 155). In examples, outer bearing surface 170 is configured to exert a reaction force on receptacle electrode 140 (e.g., receptacle electrode body 155) in a direction away from receptacle axis LR (e.g., in the first radial direction RD1) when receptacle electrode 140 exerts an action force on outer bearing surface 170 in a direction toward receptacle axis LR (e.g., in the second radial direction RD2). Outer bearing surface 170 may be configured such that the reaction force limits and/or ceases movement of receptacle electrode 140 toward receptacle axis LR such that, for example, receptacle electrode body 155 remains substantially stationary with respect to wall body 143. In examples, receptacle electrode body 155 defines an inward bearing surface 172 configured to contact and/or be in contact with outer bearing surface 170. In examples, outer bearing surface 170 and/or inward bearing surface 172 define at least some portion of a perimeter around receptacle axis LR. For example, outer bearing surface 170 and/or inward bearing surface 172 may define at least a portion of a perimeter of a cross-sectional area perpendicular to and intersected by receptacle axis LR.

Wall body 143 may define an inner bearing surface 174 configured to limit and/or constrain movement of receptacle electrode 140 with respect to wall 110 in a direction away from receptacle axis LR (e.g., in the first radial direction RD1). Inner bearing surface 174 may be configured to contact and/or be in contact with receptacle electrode 140 (e.g., receptacle electrode body 155). In examples, inner bearing surface 174 is configured to exert a reaction force on receptacle electrode 140 (e.g., receptacle electrode body 155) in a direction toward receptacle axis LR (e.g., in the second radial direction RD2) when receptacle electrode 140 exerts an action force on inner bearing surface 174 in a direction away from receptacle axis LR (e.g., in the first radial direction RD1). Inner bearing surface 174 may be configured such that the reaction force limits and/or ceases movement of receptacle electrode 140 away from receptacle axis LR such that, for example, receptacle electrode body 155 remains substantially stationary with respect to wall body 143. In examples, receptacle electrode body 155 defines an outward bearing surface 176 configured to contact and/or be in contact with inner bearing surface 174. In examples, inner bearing surface 174 and/or outward bearing surface 176 define at least some portion of a perimeter around receptacle axis LR. For example, inner bearing surface 174 and/or outward bearing surface 176 may define at least a portion of a perimeter of a cross-sectional area perpendicular to and intersected by receptacle axis LR.

In examples, receptacle electrode 140 defines inward bearing surface 172 in a first portion 178 of receptacle electrode body 155 (“first electrode portion 178”). Receptacle electrode 140 may define outward bearing surface 176 in a second portion 180 of receptacle electrode body 155 (“second electrode portion 180”). In examples, first electrode portion 178 defines at least a portion of outer electrode face 156. For example, first electrode portion 178 may define a portion of outer electrode face 156 separated from inward bearing surface 172 by a portion of receptacle electrode body 155. First electrode portion 178 may define a portion of outer electrode face 156 facing in a direction substantially away from inward bearing surface 172 (e.g., facing in the first radial direction RD1). In examples, second electrode portion 180 defines at least a portion of inner electrode face 154. For example, second electrode portion 180 may define at least a portion of inner electrode face 154 separated from outward bearing surface 176 by a portion of receptacle electrode body 155. Second electrode portion 180 may define at least a portion of inner electrode face 154 in a direction substantially away from outward bearing surface 176 (e.g., facing in the second radial direction RD2). In examples, first electrode portion 178 is configured to be distal to second electrode portion 180 when receptacle electrode 140 contacts wall body 143.

In some examples, wall body 143 defines a cross-section dimension D2 extending radially in a direction from receptacle axis LR. In example, dimension D2 is perpendicular to receptacle axis LR. Wall outer surface 142 and wall inner surface 144 may be substantially separated by dimension D3. First electrode portion 178 may define a cross-section dimension D3 extending radially in a direction from receptacle axis LR, and in in some examples, perpendicular to receptacle axis LR. Outer electrode face 156 and inward bearing surface 170 may be substantially separated by dimension D3. In some examples, dimension D3 may be parallel to dimension D2. Dimension D3 may have any magnitude relative to dimension D2. In examples, dimension D3 is greater than or equal to about 40% of dimension D2 and less than or equal to about 60% of dimension D2. In some examples, dimension D3 is less than dimension D2. In examples, dimension D3 is greater than 20%, in some examples, greater than 40%, in some examples greater than 60%, and in some examples greater than about 80%, of dimension D2.

In some examples, wall body 143 defines a cross-section dimension D2 extending radially in a direction from receptacle axis LR. In example, dimension D2 is perpendicular to receptacle axis LR. Wall outer surface 142 and wall inner surface 144 may be substantially separated by dimension D3. First electrode portion 178 may define a cross-section dimension D3 extending radially in a direction from receptacle axis LR, and in some examples, perpendicular to receptacle axis LR. Outer electrode face 156 and inward bearing surface 170 may be substantially separated by dimension D3. In some examples, dimension D3 may be parallel to dimension D2. Dimension D3 may have any magnitude relative to dimension D2. In examples, dimension D3 is greater than or equal to about 40% of dimension D2 and less than or equal to about 60% of dimension D2. In some examples, dimension D3 is less than dimension D2. In examples, dimension D3 is greater than 20%, in some examples, greater than 40%, in some examples greater than 60%, and in some examples greater than about 80%, of dimension D2.

In some examples, wall body 143 defines trapping portion 182 configured to trap at least a portion of receptacle electrode body 155 (e.g., second electrode portion 180 or first electrode portion 178) between a first section 184 of trapping portion 182 (“first trapping section 184”) and second section 186 of trapping portion 182 (“second trapping section 186”). Trapping portion 182 may be configured such that the portion of receptacle electrode body 155 positions substantially between first trapping section 184 and second trapping section 186. In examples, first trapping section 184 is configured to exert a reaction force on receptacle electrode 140 (e.g., receptacle electrode body 155) in a direction toward receptacle axis LR (e.g., in the second radial direction RD2) when receptacle electrode 140 exerts an action force on first trapping section 184 in a direction away from receptacle axis LR (e.g., in the first radial direction RD1). Second trapping section 186 may be configured to exert a reaction force on receptacle electrode 140 (e.g., receptacle electrode body 155) in a direction away from receptacle axis LR (e.g., in the first radial direction RD1) when receptacle electrode 140 exerts an action force on second trapping section 186 in a direction toward from receptacle axis LR (e.g., in the second radial direction RD2).

In examples, receptacle electrode 140 may be substantially embedded within receptacle wall 110. In examples, receptacle electrode 140 may be affixed to receptacle wall 110 using adhesives, soldering, boundary interlocking, and/or other methods. Receptacle electrode 140 may be affixed to receptacle wall 110 such that receptacle electrode 140 is limited and/or constrained from movement relative to receptacle wall 110. In some examples, receptacle electrode 140 is affixed to receptacle wall 110 by overmolding. For example, receptacle wall 110 may be fabricated by injection molding a molten polymer and/or other molten material substantially around receptacle electrode 140. The molten polymer and/or other molten material may form a mechanically interlocked boundary with a boundary of receptacle electrode 140, such as inward bearing surface 172, outward bearing surface 176, and/or one or more surfaces defined by first electrode portion 178, second electrode portion 180, trapping portion 182, and/or other surfaces of electrode 140. In some examples, receptacle electrode 140 includes one or more holding fixtures such as fixture 188, fixture 190, and/or fixture 192 configured to engage a holding tool (e.g., during an injection molding process). Fixtures 188, 190, 192 may define, for example, a recess into or a protrusion extending from outer electrode face 156, inner electrode face 154, or another surface defined by receptacle electrode body 155. Receptacle wall 110 may be fabricated by any method, including molding, machining, forging, or other methods. In examples, receptacle wall 110 comprises a polymer. In examples, receptacle electrode 140 comprises a metal or a conductive polymer.

In examples, as illustrated in, for example, FIG. 4, receptacle device 108 includes one or more standoff ridges extending toward receptacle volume 112, such as standoff ridge 194 and standoff ridge 196. Standoff ridges 194, 196 may be configured to maintain a displacement (e.g., the displacement D1 (FIGS. 7, 8)) between inner electrode face 154 and one or more of electrodes 161, such as IMD return electrode 166. In examples, standoff ridges 194, 196 are configured to allow some portion of a fluid comprising internal environment EI within receptacle volume 112 to flow into the displacement between inner electrode face 154 and the one or more of electrodes 161 (e.g., IMD return electrode 166). Standoff ridges 194, 196 may be configured to extend from wall inner surface 144 in a direction toward receptacle axis LR. In examples, standoff ridges 194, 196 are substantially adjacent inner electrode face 154. In examples, standoff ridges 195, 196 define at least some portion of a boundary separating inner electrode face 154 and one or more portions of inner wall surface 144. In some examples, standoff ridges 195, 196 define at least some portion of a boundary around inner electrode face 154 and/or second electrode portion 180 defined by wall body 143.

Outer electrode face 156 may define a width W (FIG. 3) extending at least partially over an outer perimeter (e.g., an outer circumference) defined by receptacle device 108. In examples, the outer perimeter surrounds receptacle axis LR. In some examples, the outer perimeter defines a boundary around a cross-sectional area substantially perpendicular to receptacle axis LR. In examples, outer electrode face 156 defines a first portion of the outer perimeter and wall outer surface 142 defines a second portion of the outer perimeter, such that outer electrode face 156 extends over the first portion and only partially extends over the outer perimeter. In some examples, the outer perimeter defines a curvature around receptacle axis LR, and outer electrode face 156 extends over an arc length of the curvature. In examples, the arc length is defined by a central angle having a vertex on receptacle axis LR. The central angle may define any angular displacement. For example, the central angle may define an angular displacement of at least 10 degrees, at least 30 degrees, at least 45 degrees, at least 90 degrees, at least 135 degrees, at least 180 degrees, or some other angular displacement. In some examples, outer electrode face 156 defines substantially an entirety of the outer perimeter. For example, outer electrode face 156 may substantially surround receptacle axis LR.

Referring to, for example, FIG. 3, FIG. 7, and FIG. 8, IMD 102, may comprise a pacemaker such as a leadless and/or wholly intracardiac pacemaker. One or more of electrodes 161 may be electrically connected to processing circuitry 134. Processing circuitry 134 may be operable connected to operating circuitry configured to deliver therapy to a patient and/or sense physiological signals of the patient using electrodes 161. In examples, as discussed, at least a portion of processing circuitry 134 (e.g., processing circuitry 133) may be supported by IMD housing 160. In examples, at least a portion of operating circuitry 134 (e.g., processing circuitry 131) may be supported by another device displaced from IMD 102, such as external device 136, and/or another device within the patient or extracorporeal to the patient.

Processing circuitry 134 may include fixed function circuitry and/or programmable operating circuitry. In examples, processing circuitry 134 includes circuitry configured to perform one or more functions of operating circuitry, such as therapy delivery circuitry, sensing circuitry, processing circuitry, switching circuitry, communication circuitry, and/or other circuitries. Processing circuitry 134, as well as other processors, operating circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 134 includes multiple components, such as any combination of one or more microprocessors, one or more DSPs, one or more ASICs, or one or more FPGAs, as well as other discrete or integrated logic circuitry, and/or analog circuitry.

Functions attributed to processing circuitry 134 may be embodied as software, firmware, hardware or any combination thereof. Processing circuitry 134 may include, for instance, a variety of capacitors, transformers, switches, and the like configured to perform the functions of processing circuitry 134. In examples, processing circuitry 134 may be configured to communicate with another device, such as a patient input/output device, a clinician input/output device, and/or others. Processing circuitry 134 may include any suitable hardware, firmware, software or any combination thereof for communicating with another device. In addition, processing circuitry 134 may communicate with a networked computing device and a computer network. In examples, processing circuitry 134 and/or other circuitry of medical system 100 is configured to deliver stimulation signals to and/or receive sensing signals from electrodes 161, delivery system electrode 158, and/or other electrodes and/or sensors within medical system 100 or external to medical system 100. Processing circuitry 134 may be configured to provide electrical signals, e.g., pacing therapy, to electrodes 161, delivery system electrode 158, and/or other electrodes within medical system 100. Processing circuitry 134 may be configured to receive electrical signals, e.g., sensed cardiac electrical signals, from electrodes 161, delivery system electrode 158, and/or other electrodes within medical system 100.

Medical system 100 (e.g., processing circuitry 134) can also include memory configured to store program instructions, such as software, which may include one or more program modules, which are executable by processing circuitry 134. The program instructions may be embodied in software and/or firmware. The memory can include any volatile, non-volatile, magnetic, optical, or electrical media, such as a random access memory (RAM), read-only memory (ROM), non-volatile RAM (NVRAM), electrically-erasable programmable ROM (EEPROM), ferroelectric RAM (FRAM), flash memory, or any other digital media. In some examples, the memory includes computer-readable instructions that, when executed by processing circuitry 134 cause processing circuitry 134 to perform various functions described herein and/or other functions of processing circuitry 134.

IMD housing 160 may enclose processing circuitry 134 and/or other circuitry within medical system 10. IMD housing 160 may be configured to fluidly isolate processing circuitry 134 and/or other circuitry from an environment in contact with an exterior surface of IMD housing 160. In examples, IMD housing 160 is configured to hermetically seal an enclosure defined by IMD 102 and holding processing circuitry 134 and/or other circuitry. IMD housing 160 may be configured to define shapes that are easily accepted by the patient's body while minimizing patient discomfort. For example, IMD housing 160 may define a substantially cylindrical shape with cylindrical sidewalls. In other examples, IMD housing 160 may define substantially rectangular or other non-cylindrical shapes. IMD housing 160 may define shapes in which corners and edges are designed with relatively large radii, in order to present a housing having smoothly contoured exterior surfaces. In examples, attachment member 132 is coupled to IMD housing 160.

Communication links 135, 137 may be hard-line and/or wireless communications links. In some examples, communication links 135, 137 may comprise some portion of control circuitry 134. In some examples, communication links 135, 137 comprise a wired connection, a wireless Internet connection, a direct wireless connection such as wireless LAN, Bluetooth™, Wi-Fi™, and/or an infrared connection. Communication links 135, 137 may utilize any wireless or remote communication protocol.

As used here, when a first portion of a system (e.g., medical system 100) is substantially parallel to a second portion of or an axis defined by the system, this may mean the first portion is parallel or nearly parallel to the second portion or the axis to the extent permitted by manufacturing tolerances. In some examples, when the first portion is substantially parallel to the second portion or the axis, this may mean a first vector defined by the first component of the system defines an angle of less than 10 degrees, in some examples less than 5 degrees, and in some examples less than 1 degree, with a second vector defined by the second component or the axis. When a first portion of the system is substantially perpendicular to a second portion of or an axis defined by the system, this may mean the first portion is perpendicular or nearly perpendicular to the second portion or the axis to the extent permitted by manufacturing tolerances. In some examples, when the first portion is substantially perpendicular to the second portion or the axis, this may mean that the first vector defined by the first component of the system defines an angle of at least 80 degrees, in some examples at least 85 degrees, and in some examples at least 89 degrees, with the second vector defined by the second component.

As used here, when a first portion of a system (e.g., medical system 100) supports a second portion of the system, this means that when the second portion causes a first force to be exerted on the first portion, the first portion causes a second force to be exerted on the second portion in response to the first force. The first force and/or second force may be a contact force and/or an action-at-a-distance force. For example, first force and/or second force may be mechanical force, a magnetic force, a gravitational force, or some other type of force. The first portion of the system may be a portion of the system or a portion of a component of the system. The second portion of the system may be another portion of the system or another portion of the same component or a different component. In some examples, when the first portion of the system supports the second portion of the system, this may mean the second portion is mechanically supported by and/or mechanically connected to the first portion.

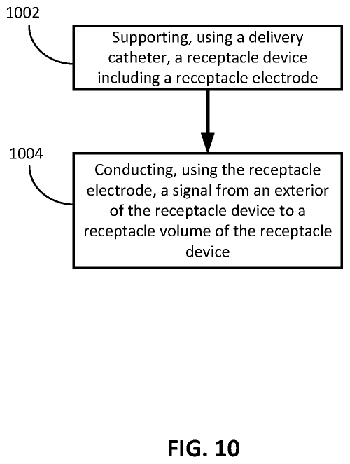

A technique for conducting a signal to a receptacle volume of a receptacle device is illustrated in FIG. 10. Although the technique is described mainly with reference to medical system 100 of FIGS. 1-9, the technique may be applied to other medical systems in other examples.

The technique includes supporting, using a delivery catheter 106, a receptacle device 108 including a receptacle electrode 140 (802). Delivery catheter 106 may support receptacle device 108 within an anatomical volume of a patient, such as a heart chamber of heart 101. In examples, receptacle device 108 defines a receptacle volume 112 holding and/or supporting an IMD 102. In examples, a delivery system 120 engages IMD 102. In examples, delivery system 120 (e.g., a delivery system body distal portion 124) extends through a delivery lumen 118 of delivery catheter 106. Delivery system body distal portion 124 may extend through delivery lumen 118 and into receptacle volume 112 via a delivery lumen opening 153. In examples, a head portion 128 supported by delivery system body distal portion engages IMD 102.

The technique includes conducting, using receptacle electrode 140, a signal (e.g., an intrinsic cardiac signal) from an external environment EO to receptacle volume 112 (804). In examples, receptacle electrode 140 conducts the signal to an internal environment EI within receptacle volume 112. External environment EO may be in fluid communication with internal environment EI. In examples, external environment EO fluidically communicates with internal environment EI at least via a receptacle opening 109 defined by and opening into receptacle volume 112.

Delivery catheter 106 may position receptacle device 108 and/or IMD 102 in proximity to a target site 104. In examples, delivery catheter 106 transports and/or guides receptacle device 108 and/or IMD 102 through vasculature of the patient. Delivery system body 122 may slidably translate and/or rotate within delivery lumen 118 to impart (e.g., via head portion 128) translational and/or rotational forces on IMD 102, such that IMD 102 translates and/or rotates relative to receptacle wall 110 within receptacle wall 112. IMD 102 may translate in the proximal direction P or the distal direction D, and or rotate about a device axis LD, relative to receptacle wall 110. IMD 102 may contact, anchor to, and/or otherwise engage a vessel wall 168 using an attachment member 132. Delivery catheter 106 may impart a translation force and/or rotational torque on IMD 102 (e.g., via head portion 128) to cause attachment member 132 to engage vessel wall 168. Delivery catheter 106 may impart a translation force and/or rotational torque on IMD 102 (e.g., via head portion 128) to cause attachment member 132 to disengage from vessel wall 168. Delivery system body 122 and/or head portion 128 may disengage from IMD 102 when attachment member 132 anchors IMD 102 to vessel wall 168. Delivery catheter 106, receptacle device 108, and delivery system 120 may be withdrawn from the patient (e.g., by a clinician) via vasculature of the patient.

Medical system 100 may receive (e.g., sense) an indication of an intrinsic cardiac electrical signal produced by heart 101 using receptacle electrode 140. Processing circuitry 134 may process and/or condition the signal sensed using at least a first electrode (e.g., one of a cathode or an anode) and a second electrode (e.g., the other of the cathode and the anode) in electrical communication with processing circuitry 134. In some examples, the first electrode or second electrode is a delivery system electrode 158 mechanically supported by delivery system body 122 and/or head portion 128. In some examples, the first electrode or second electrode is one of device electrodes 161 (e.g., IMD distal electrode 162, IMD atrial electrode 164, and/or IMD return electrode 166) mechanically supported by a IMD housing 160, or one or more electrodes mechanically supported by another portion of medical system 100. In some examples, the first electrode or second electrode is an external electrode 138 extracorporeal to the patient and/or otherwise displaced from receptacle device 108 and/or IMD 102 (e.g., a cutaneous electrode affixed to the skin of the patient).

Receptacle electrode 140 may provide a conductive path through receptacle wall 110 to reduce attenuation, shielding, and/or interference as a signal travels over a signal path from external environment EO to an electrode within receptacle volume 112. For example, receptacle electrode 140 may provide the conductive path through receptacle wall 110 to provide a signal path from external environment EO to delivery system electrode 158. Delivery system electrode 158 may communicate the signal to device processing circuitry 131. Device processing circuitry 131 may be mechanically supported by an external device 136 extracorporeal to the patient and/or otherwise displaced from receptacle device 108 and/or IMD 102. Medical system 100 use device processing circuitry 131, delivery system electrode 158, and another electrode such as external electrode 138 to assess whether receptacle device 108 is within an atrium of heart 101, a ventricle of heart 101, a coronary sinus of heart 101, or some other portion of heart 101.

Receptacle electrode 140 may provide the conductive path when IMD 102 is in a first position (e.g., a delivery position) within receptacle volume 112 (e.g., as depicted in FIG. 7).

Receptacle electrode 140 may provide the conductive path through receptacle wall 110 to provide a signal path from external environment EO to IMD return electrode 166. IMD return electrode 166 may communicate the signal to IMD processing circuitry 133 mechanically supported by IMD housing 160. Medical system 100 may use IMD processing circuitry 133, IMD return electrode 166, and another electrode such as IMD distal electrode 162, IMD atrial electrode 164, and/or another of electrodes 161 to assess a proximity of IMD 102 (e.g., attachment member 132) to vessel wall 168 of heart 101, conduct pace mapping to determine a suitable placement of IMD 102, evaluate a suitability of pacing delivered by IMD 102 at a particular location, and/or for other reasons. Receptacle electrode 140 may provide the conductive path when IMD 102 is in the second position within receptacle volume 112 (e.g., as depicted in FIG. 8).

Various examples of the disclosure have been described. Any combination of the described systems, operations, or functions is contemplated. These and other examples are within the scope of the following claims.

Example 1. A medical system comprising: a receptacle device including a receptacle wall defining a receptacle volume configured to receive at least a portion of an implantable medical device, wherein the receptacle device is configured to be positioned within an anatomical volume defined by a body of a patient, and wherein the receptacle device includes an receptacle electrode configured to conduct a signal from an environment surrounding an exterior of the receptacle device to the receptacle volume; and a delivery catheter supporting the receptacle device, wherein the delivery catheter defines a delivery lumen and a delivery lumen opening which opens into the receptacle volume.

Example 2. The medical system of Example 1, wherein the receptacle wall defines an inner surface defining the receptacle volume and an outer surface opposite the inner surface, wherein the receptacle electrode extends from the outer surface to the inner surface.

Example 3. The medical system of Example 1 or Example 2, wherein the receptacle electrode is embedded within the receptacle wall.

Example 4. The medical system of any of Examples 1-3, wherein the receptacle wall at least partially surrounds a receptacle axis defined by the receptacle device and extending through the receptacle volume, and wherein the receptacle wall defines one or more support surfaces configured to limit movement of the receptacle electrode in at least one of a direction radial to the receptacle axis or a direction parallel to the receptacle axis.

Example 5. The medical system of any of Examples 1-4, wherein the delivery catheter is attached to a proximal portion of the receptacle device, wherein a distal end of the receptacle device defines a receptacle opening which opens into the receptacle volume, and wherein the receptacle opening is configured to allow the implantable medical device to pass therethrough.

Example 6. The medical system of any of Examples 1-5, wherein the receptacle wall is configured to establish a displacement between the receptacle electrode and a device electrode of the implantable medical device when the receptacle volume receives the portion of the implantable medical device.

Example 7. The medical system of Example 6, wherein the receptacle wall at least partially surrounds a receptacle axis defined by the receptacle device and extending through the receptacle volume, and wherein the receptacle wall defines a standoff ridge extending toward the receptacle axis, wherein the standoff ridge is configured to establish the displacement between the receptacle electrode and the device electrode when the receptacle volume receives the portion of the implantable medical device.

Example 8. The medical system of any of Examples 1-5, wherein the receptacle electrode is configured to contact a device electrode of the implantable medical device when the receptacle volume receives the portion of the implantable medical device.

Example 9. The medical system of any of Examples 1-8, wherein the receptacle wall comprises a first material having a first conductivity and the receptacle electrode comprises a second material having a second conductivity, wherein the second conductivity is greater than the first conductivity.

Example 10. The medical system of any of Examples 1-9, wherein the receptacle electrode defines an inner electrode face facing toward the receptacle volume and an outer electrode face facing away from the receptacle volume, the outer electrode face configured to receive the signal from the environment surrounding the exterior of the receptacle device, wherein the receptacle electrode configured to conduct the signal from the outer electrode face to the inner electrode face.

Example 11. The medical system of Example 10, wherein: a distal end of the receptacle device defines a receptacle opening which opens into the receptacle volume, the receptacle wall defines a midpoint halfway between the delivery lumen opening and the receptacle opening, and the outer electrode face extends distal to the midpoint.

Example 12. The medical system of Example 10 or Example 11, wherein the inner electrode face extends more proximally than the outer electrode face.

Example 13. The medical system of any of Examples 1-12, wherein the receptacle device defines an outer perimeter at least partially surrounding a receptacle axis defined by the receptacle device and extending through the receptacle volume, and wherein the receptacle electrode extends at least partially around the outer perimeter.

Example 14. The medical system of Example 13, wherein the outer perimeter surrounds the receptacle axis, and wherein the receptacle electrode extends around the outer perimeter.

Example 15. The medical system of any of Examples 1-14, further comprising a delivery system including a delivery system body, wherein: a distal portion of the delivery system body is configured to extend through the delivery lumen and into the receptacle volume, the distal portion of the delivery system is configured to engage with the implantable medical device, and the delivery system body is configured to slidably translate within the delivery lumen to cause the implantable medical device to translate within the receptacle volume in a distal direction or a proximal direction relative to the receptacle wall.

Example 16. The medical system of Example 15, wherein the delivery system body includes a delivery system electrode configured to receive the signal from the receptacle electrode when the distal portion of the delivery system body extends through the delivery lumen.

Example 17. The medical system of Example 16, wherein the delivery system is configured to communicate the signal from the delivery system electrode to processing circuitry configured to receive the signal from the device electrode when the distal portion of the delivery system body extends through the delivery lumen.

Example 18. The medical system of any of Examples 15-17, wherein the delivery system body is configured to rotate relative to the receptacle wall when the distal portion of the delivery system body extends through the delivery lumen.

Example 19. The medical system of any of Examples 1-18, further comprising the implantable medical device, wherein the implantable medical device comprises a device electrode configured to receive the signal from the receptacle electrode when the implantable medical device is positioned within the receptacle volume.

Example 20. The medical system of Example 19, wherein a housing of the implantable medical device mechanically supports the device electrode.

Example 21. The medical system of Example 19 or Example 20, wherein the implantable medical device mechanically supports processing circuitry, and wherein the device electrode is configured to communicate the signal to the processing circuitry when the device electrode receives the signal from the receptacle electrode.

Example 22. The medical system of any of Examples 19-21, wherein the implantable medical device defines a device axis extending from a proximal portion of the implantable medical device to a distal portion of the implantable medical device.

Example 23. The medical system of Example 22, wherein the device electrode extends at least partially around the device axis.

Example 24. The medical system of Example 22 or Example 23, wherein the distal device portion includes an attachment member configured to engage tissues.

Example 25. The medical system of any of Examples 22-24, wherein the proximal portion of the implantable medical device is configured to mechanically engage a delivery system extending through the delivery lumen.

Example 26. The medical system of any of Examples 1-25, wherein the anatomical volume is a chamber of a heart of the patient.

Example 27. The medical system of any of Examples 1-26, wherein the delivery catheter is configured transport the receptacle device through vasculature of the patient.

Example 28. A method, comprising: supporting, using a delivery catheter, a receptacle device, wherein the receptacle device includes a receptacle wall defining a receptacle volume configured to receive at least a portion of an implantable medical device, and wherein the delivery catheter defines a delivery lumen and a delivery lumen opening which opens into the receptacle volume; and conducting, using a receptacle electrode of the receptacle wall, a signal from an environment surrounding an exterior of the receptacle device to the receptacle volume.

Example 29. The method of Example 28, further comprising conducting, using the electrode, the signal through a conductive path defined by the electrode and extending through the receptacle wall.

Example 30. The method of Example 28 or Example 29, further comprising limiting, using one or more support surfaces of the receptacle wall, movement of the receptacle electrode in at least one of a direction radial to a receptacle axis or a direction parallel to the receptacle axis, wherein the receptacle device defines the receptacle axis and the receptacle axis extends through the receptacle volume.

Example 31. The method of any of Examples 28-30, further comprising passing, using a delivery system extending through the delivery lumen, the implantable medical device through a receptacle opening which opens into the receptacle volume.

Example 32. The method of any of Examples 28-31, further comprising establishing, using the receptacle wall, a displacement between the receptacle electrode and a device electrode of the implantable medical device when the receptacle volume receives the portion of the implantable medical device.

Example 33. The method of Example 32, further comprising establishing the displacement using a standoff ridge defined by the receptacle wall, wherein the ridge extends toward a receptacle axis defined by the receptacle device and extending through the receptacle volume.

Example 34. The method of any of Examples 28-31, further comprising contacting, using the receptacle electrode, a device electrode of the implantable medical device when the receptacle volume receives the portion of the implantable medical device.

Example 35. The method of any of Examples 28-34, further comprising conducting, using a receptacle electrode, the signal through a first material having a first conductivity, wherein the first conductivity is greater than a second conductivity of a second material comprising the receptacle wall.

Example 36. The method of any of Examples 28-35, further comprising conducting, using the receptacle electrode, the signal from an outer electrode face facing away from the receptacle volume to an inner electrode face facing toward the receptacle volume.

Example 37. The method of any of Examples 28-36, further comprising: engaging, using a delivery system extending through the delivery lumen, the implantable medical device; and translating, using the delivery system, the implantable medical device within the receptacle volume in a distal direction or a proximal direction relative to the receptacle wall.

Example 38. The method of any of Examples 28-37, further comprising receiving, using a delivery system electrode of a delivery system extending through the delivery lumen, the signal from the receptacle electrode.

Example 39. The method of Example 38, further comprising communicating, using the delivery system, the signal from the delivery system electrode to processing circuitry configured to receive the signal from the delivery system electrode.

Example 40. The method of any of Examples 28-39, further comprising rotating, using a delivery system extending through the delivery lumen, the implantable medical device relative to the receptacle wall.

Example 41. The method of any of Examples 28-39, further comprising receiving, using a device electrode of the implantable medical device, the signal from the receptacle electrode.

Example 42. The method of Example 41, further comprising communicating, using the implantable medical device, the signal from the device electrode to the processing circuitry mechanically supported by a housing of the implantable medical device.

Example 43. The method of any of Examples 28-42, further comprising engaging, using an attachment member of the implantable medical device, tissues of the patient.

Example 44. The method of any of Examples 28-43, further comprising transporting, using the delivery catheter, the receptacle device through vasculature of the patient.

1: A medical system comprising:
a receptacle device including a receptacle wall defining a receptacle volume configured to receive at least a portion of an implantable medical device,
wherein the receptacle device is configured to be positioned within an anatomical volume defined by a body of a patient, and
wherein the receptacle device includes a receptacle electrode configured to conduct a signal from an environment surrounding an exterior of the receptacle device to the receptacle volume; and
a delivery catheter supporting the receptacle device, wherein the delivery catheter defines a delivery lumen and a delivery lumen opening which opens into the receptacle volume.

2: The medical system of claim 1, wherein the receptacle wall defines an inner surface defining the receptacle volume and an outer surface opposite the inner surface, wherein the receptacle electrode extends from the outer surface to the inner surface.

3: The medical system of claim 1, wherein the receptacle electrode is embedded within the receptacle wall.

4: The medical system of claim 1, wherein the receptacle wall at least partially surrounds a receptacle axis defined by the receptacle device and extending through the receptacle volume, and wherein the receptacle wall defines one or more support surfaces configured to limit movement of the receptacle electrode in at least one of a direction radial to the receptacle axis or a direction parallel to the receptacle axis.

5: The medical system of claim 1,
wherein the delivery catheter is attached to a proximal portion of the receptacle device, wherein a distal end of the receptacle device defines a receptacle opening which opens into the receptacle volume, and
wherein the receptacle opening is configured to allow the implantable medical device to pass therethrough.

6: The medical system of claim 1, wherein the receptacle wall is configured to establish a displacement between the receptacle electrode and a device electrode of the implantable medical device when the receptacle volume receives the portion of the implantable medical device.

7: The medical system of claim 1, wherein the receptacle electrode defines an inner electrode face facing toward the receptacle volume and an outer electrode face facing away from the receptacle volume, the outer electrode face configured to receive the signal from the environment surrounding the exterior of the receptacle device, wherein the receptacle electrode configured to conduct the signal from the outer electrode face to the inner electrode face.

8: The medical system of claim 7, wherein:
a distal end of the receptacle device defines a receptacle opening which opens into the receptacle volume,
the receptacle wall defines a midpoint halfway between the delivery lumen opening and the receptacle opening, and
the outer electrode face extends distal to the midpoint.

9: The medical system of claim 7, wherein the inner electrode face extends more proximally than the outer electrode face.

10: The medical system of claim 1, wherein the receptacle device defines an outer perimeter at least partially surrounding a receptacle axis defined by the receptacle device and extending through the receptacle volume, and wherein the receptacle electrode extends at least partially around the outer perimeter.

11: The medical system of claim 1, further comprising a delivery system including a delivery system body, wherein:
a distal portion of the delivery system body is configured to extend through the delivery lumen and into the receptacle volume, the distal portion of the delivery system is configured to engage with the implantable medical device, and
the delivery system body is configured to slidably translate within the delivery lumen to cause the implantable medical device to translate within the receptacle volume in a distal direction or a proximal direction relative to the receptacle wall.

12: The medical system of claim 11, wherein the delivery system body includes a delivery system electrode configured to receive the signal from the receptacle electrode when the distal portion of the delivery system body extends through the delivery lumen.

13: The medical system of claim 1, further comprising the implantable medical device, wherein the implantable medical device comprises a device electrode configured to receive the signal from the receptacle electrode when the implantable medical device is positioned within the receptacle volume,
wherein the implantable medical device mechanically supports processing circuitry, and
wherein the device electrode is configured to communicate the signal to the processing circuitry when the device electrode receives the signal from the receptacle electrode.

14: A method, comprising:
supporting, using a delivery catheter, a receptacle device, wherein the receptacle device includes a receptacle wall defining a receptacle volume configured to receive at least a portion of an implantable medical device, and wherein the delivery catheter defines a delivery lumen and a delivery lumen opening which opens into the receptacle volume; and
conducting, using a receptacle electrode of the receptacle wall, a signal from an environment surrounding an exterior of the receptacle device to the receptacle volume.

15: The method of claim 14, further comprising conducting, using the receptacle electrode, the signal from an outer electrode face facing away from the receptacle volume to an inner electrode face facing toward the receptacle volume.

16: The method of claim 14, further comprising:
establishing, using the receptacle wall, a displacement between the receptacle electrode and a device electrode of the implantable medical device when the receptacle volume receives the portion of the implantable medical device.

17: The method of claim 16, further comprising:
establishing the displacement using a standoff ridge defined by the receptacle wall, wherein the standoff ridge extends toward a receptacle axis defined by the receptacle device and extending through the receptacle volume.

18: The method of claim 14, further comprising:
engaging, using a delivery system extending through the delivery lumen, the implantable medical device; and
translating, using the delivery system, the implantable medical device within the receptacle volume in a distal direction or a proximal direction relative to the receptacle wall.

19: The medical system of claim 1, wherein the receptacle electrode is configured to contact a device electrode of the implantable medical device when the receptacle volume receives the portion of the implantable medical device.

20: The medical system of claim 1, wherein the receptacle wall comprises a first material having a first conductivity and the receptacle electrode comprises a second material having a second conductivity, wherein the second conductivity is greater than the first conductivity.
